(19) United States
(12) Patent Application Publication
(10) Pub. No.: US 2024/0355462 A1
(43) Pub. Date: Oct. 24, 2024

(54) ARTIFICIAL INTELLIGENCE PATIENT INTAKE AND APPOINTMENT BOOKING SYSTEM

(71) Applicant: Lightning Comm LLC, Montebello, NY (US)

(72) Inventors: Joshua L. Fox, Lawrence, NY (US); Jacob Baldinger, Monsey, NY (US)

(21) Appl. No.: 18/641,464

(22) Filed: Apr. 22, 2024

Related U.S. Application Data
(60) Provisional application No. 63/497,440, filed on Apr. 21, 2023.
(60) Provisional application No. 63/537,554, filed on Sep. 11, 2023.

(51) Int. Cl. G16H 40/20; G06N 20/00; G06Q 10/02; G10L 15/22

(52) U.S. Cl. CPC G16H 40/20; G06N 20/00; G06Q 10/02; G10L 15/22

(57) ABSTRACT

A solution for patient scheduling systems, designed to streamline the appointment booking process and enhance the overall patient experience is provided. The solution may include a kiosk computer system that interacts with a patient using voice recognition technology. The solution may include an appointment booking system. The appointment booking system may include an interactive voice response system, a middleware system and an electronic health record system. The system may also include an artificial intelligence model that is less susceptible to hallucinations. provide a seamless and efficient appointment booking process. The solution may adapt to office-specific requirements and improve patient satisfaction and practice efficiency.

### CROSS-REFERENCE TO RELATED APPLICATIONS

This application is a nonprovisional of, and claims priority to U.S. Provisional Patent Application No. 63/497,440, filed on Apr. 21, 2023, and U.S. Provisional Patent Application No. 63/537,554, filed on Sep. 11, 2023, both of which are hereby incorporated by reference herein in their entireties.

### FIELD OF TECHNOLOGY

The present disclosure relates to a system for improving workflow in a healthcare facility. The system leverages improved artificial intelligence (“AI”) and multiple communication channels to provide patients with a dynamic, intuitive and efficient intake experience.

### BACKGROUND

Medical offices are notoriously hard to reach. Unless you have an urgent health problem that demands prompt attention, an appointment is usually required to be seen by a doctor or get other medically related services. Scheduling an appointment can be very frustrating. Many patients report “operational friction” which includes long hold times, difficulty getting a timely appointment and trouble accessing follow-up information. Challenges scheduling a medical appointment are further exacerbated because, despite a proliferation of online scheduling tools, more than half of all medical appointments are still scheduled over the phone. In 2022, telephone calls were the most preferred communication channel when a consumer needs help for any industry, an 8% increase since 2021.

Healthcare consumers are even more likely to pick up the phone. The majority of healthcare patients start their journey by searching for and comparing providers online. However, after completing their research, patients typically attempt to schedule an appointment by calling. Although phone calls are highly preferred by patients, they are also one of the most frustrating communication channels. A patient may need to speak with multiple people and suffer interminable hold time. The front desk staff at a medical office are often juggling multiple tasks. They need to help book or change appointments, authenticate scheduled patients, verify insurance information, physically guide patients into the exam room. The staff is also needs to field general questions regarding billing, payments, pharmacy prescriptions and other questions.

Conventional interactive voice response (“IVR”) systems do not provide any relief to the operational friction. Interactive voice response, or IVR, is an automated telephone system technology that enables callers to receive or provide information, or make requests using voice or menu inputs, without speaking to a live agent. IVR is powered by a pre-recorded messaging or text-to-speech technology with a dual-tone multi-frequency (DTMF) interface.

A conventional IVR system is programmed to handle a predefined set of tasks and often does not provide flexibility for handling other tasks. Consumers often feel frustrated when interacting with conventional IVR systems. Within conventional IVR systems, it is difficult to directly access a live agent. Consumers cannot proceed directly to the task they need and typically waste time listening to irrelevant menu options. Finally, conventional IVR systems have difficulty understanding voice inputs received from consumers. Typically, consumers cannot interact with the conventional IVR system using complete sentences expressed in natural language, as consumers would do when speaking to a live agent.

Recent advances in artificial intelligence (“AI”) have increased optimism that IVR systems may be able to provide a better consumer experience. For example, conversational AI leverages large volumes of data, machine learning and natural language processing to help imitate human interactions, recognizing speech and text inputs and translating their meanings across various languages. However, conversational AI systems also have several technological deficiencies.

Conversational AI systems are designed to collect information required to complete a task. This information can come from contextual information or asking the caller a question. Every question the AI asks the caller is an opportunity for the conversation to fail. For example, the caller may need to provide an 11-digit ID number, an address or name for the automated system to accurately identify the caller. In voice environments, it may be challenging to correctly capture any of these data elements, even if the caller knows them.

AI models are also vulnerable to hallucinations. A hallucination is a phenomenon when an AI model perceives patterns or inputs that are nonexistent or imperceptible to humans, creating outputs that are nonsensical or inaccurate. Preventing hallucinations is difficult. Some notable examples of AI hallucination include:
Incorrect predictions, such as predicting that an event will occur when it is unlikely to happen. An AI model used to predict the weather may predict that it will rain tomorrow when there is no rain in the forecast.
False positives, such as identifying something as being a threat or a fact when it is not. An AI model may concoct nonexistent company return, refund or cancellation policies.
False negatives, such as failing to identify something as being a threat when it is. An AI model used to detect cancer may fail to identify a cancerous tumor.

These hallucinations, which at times may be comical, are serious concerns for mission-critical applications such healthcare. Healthcare applications require reliable and consistent outputs. This need has created a technological challenge. On the one hand, patients want and need automated solutions to improve access to and delivery of healthcare services. On the other hand, conventional automated solutions are unable to interact with patients in a natural and conversational way. Current AI solutions are not sufficiently reliable for healthcare applications.

Accordingly, it would be desirable to provide an improved AI solution that is less prone to hallucinations and allows patients to interact with automated systems using conversational, natural language. Accordingly, it is desirable to provide apparatus and methods for an ARTIFICIAL INTELLIGENCE PATIENT INTAKE AND APPOINTMENT BOOKING SYSTEM.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows an illustrative system architecture in accordance with the principles of the description;

FIG. 2 shows an illustrative process in accordance with the principles of the description;

FIG. 3 shows an illustrative process and system components in accordance with the principles of the description;

FIG. 4 shows an illustrative process and system components in accordance with the principles of the description;

FIG. 5 shows an illustrative process and system components in accordance with the principles of the description;

FIG. 6 shows an illustrative system architecture in accordance with the principles of the description;

FIG. 7 shows an illustrative graphical user interface in accordance with the principles of the description;

FIG. 8 shows an illustrative graphical user interface in accordance with the principles of the description;

FIG. 9 shows an illustrative graphical user interface in accordance with the principles of the description;

FIG. 10 shows an illustrative graphical user interface in accordance with the principles of the description;

FIG. 11 shows an illustrative kiosk in accordance with the principles of the description;

FIG. 12 shows an illustrative kiosk in accordance with the principles of the description;

FIG. 13 shows an illustrative kiosk in accordance with the principles of the description;

FIG. 14 shows an illustrative kiosk in accordance with the principles of the description;

FIG. 15 shows an illustrative kiosk in accordance with the principles of the description; and

FIG. 16 shows an illustrative kiosk in accordance with the principles of the description.

### DETAILED DISCLOSURE

AI powered IVR technology has numerous practical healthcare applications. AI powered IVR systems may efficiently implement pre-treatment questionnaires, patient satisfaction surveys, lab and appointment scheduling, appointment follow-up and patient monitoring. However, in the context of healthcare application such AI powered solutions must be more reliable and less susceptible to hallucinations.

Hallucinations are more likely when callers are able to freely provide input in a natural language conversational way. Hallucination risk may be reduced if the options for interacting with the AI are limited or scripted. However, such interaction limits cause automated systems to be less conversational and function similar to conventional IVR systems. Apparatus and methods are provided for a system that reduces hallucinations in software that incorporates an AI model. Apparatus may include one or more features of a computer system.

Apparatus may include an IVR system. The IVR system may include one or more features of a computer system. The IVR system may be configured to receive a voice input from a caller. The voice input may be a natural language statement of the caller. The IVR system may be configured to compute a first intent of the caller from the voice input. An “intent” of a caller provided voice input may refer to a recognition or understanding of what a caller wants to accomplish. Understanding a caller's intent allows an IVR system to provide personalized and meaningful responses. By providing accurate and reliable intent recognition, an IVR system does not need to offer callers a fixed menu of options. Instead, the IVR system can ask open-ended, natural language questions such as, “How can I help you today?”

Computing an intent may include determining how a caller's voice input relates to content of a prompt presented by the IVR system. If the voice input includes a number is that number part of an address, a date or an amount? Did the caller say “to,” “too,” or “two?” The relationship between caller input and the IVR prompt may be referred to as “entity recognition.”

Computing an intent may include determining contextual awareness for a caller's voice input. Contextual awareness may be determined based on prior system interactions with a caller. For example, callers having a target demographic profile may desire appointments having a template. The template may include a target date, time, provider or location. When a caller accesses the IVR system that fits the demographic profile, the IVR system may automatically greet the caller and offer the earliest available appointment that fits the template.

Apparatus may include an AI model. The AI model may include one or more features of a computer system. The AI model may utilize predictive analytics to compute contextual awareness. By analyzing past call data, an AI model may detect patterns, such as peak call times or common customer queries. The detection of these patterns may allow the AI model to make prediction and proactive adjustments to an IVR system. The predictions and adjustments may allow the IVR system to provide faster response times and reduced wait times for callers. For example, the AI model may predict patterns, such as peak call times or common customer queries and activate an IVR system to assist answering calls during those times.

Computing an intent may include determining fulfillment—a service that fulfills the caller's needs. Fulfillment may include linking disparate data sources to provide a service or respond to a caller's request. Illustrative disparate data sources may include financial, health, employee, product, enterprise, inventory, weather, or any number of internal and external data sources.

The IVR system may include an AI model. The AI model may be provided by a commercial vendor, such models and services provided by OpenAI, L.L.C. located at 3180 18th St. San Francisco, CA 94110 (e.g., ChatGPT or InstructGPT) or those provided by Google LLC located at 1600 Amphitheatre Parkway Mountain View, California 94043 USA (e.g., BARD).

An AI model may utilize one or more machine learning algorithms. An illustrative machine learning algorithm may include linear regression. Linear regression is a statistical method that models a relationship between a dependent variable and one or more independent variables. The linear regression algorithm is configured to compute a straight line that best fits between multiple data points. Linear regression is widely used for forecasting, trend analysis and risk assessment in various fields.

An illustrative machine learning algorithm may include logistic regression. Similar to linear regression, logistic regression is typically used to model a relationship between a dependent variable and one or more independent variables. However, the dependent variable in logistic regression is categorical, meaning it can only take on a limited number of values (e.g., 0 or 1, yes or no). This makes it useful for classification tasks, such as spam filtering or predicting customer churn.

An illustrative machine learning algorithm may include decision trees. The decision trees algorithm uses a tree-like structure to classify data. The algorithm asks a series of questions about data, and based on the answers, follows a branch in the tree that leads to a final classification. Decision tree algorithms are popular for their interpretability, because it is generally relatively easy to understand the logic behind their decisions.

An illustrative machine learning algorithm may include naive bayes. The naive bayes algorithm is part of a family of generative machine learning algorithms. The naive bayes algorithm is based on the Bayes theorem, which calculates the probability of an event happening given some evidence. The naive bayes algorithm provides an efficient model that works well for many classification tasks, especially when dealing with large datasets.

An illustrative machine learning algorithm may include a K-Nearest Neighbors (KNN) algorithm. The KNN algorithm is a versatile algorithm for classification and regression tasks. It operates by classifying a new data point based on the labels of its nearest neighbors in the training data. The number of neighbors (K) is a parameter that can be tuned to improve the performance of the model.

An illustrative machine learning algorithm may include support vector machines (SVM). SVMs are a powerful classification algorithm that can be used for a variety of tasks, including image recognition, natural language processing, and bioinformatics. SVMs work by finding a geometric hyperplane that best separates the data points of different classes. A hyperplane is a generalization of a two-dimensional plane in three-dimensional space to mathematical spaces of arbitrary dimension. SVMs aim to find the best hyperplane that maximizes the margin between support vectors, enabling effective classification even in complex, non-linear scenarios.

An illustrative machine learning algorithm may include deep neural networks (DNNs). DNN are modeled based on the structure of the human brain and are capable of learning complex patterns from data. DNNs learn by progressively transforming the input data through multiple layers of interconnected neurons. The weights in these connections are adjusted during training to achieve the desired output. DNNs are typically used for tasks such as image recognition, speech recognition, and machine translation.

An illustrative machine learning algorithm may include random forests. Random forest is an ensemble learning algorithm that combines multiple decision trees to create a robust and accurate AI model. The random forest algorithm takes a random sample of features at each split in the decision tree, which helps to reduce overfitting. Random forests are popular for classification tasks, especially when dealing with high-dimensional data.

An illustrative machine learning algorithm may include gradient boosting. Like random forest, gradient boosting is also an ensemble learning method. Gradient boosting builds a model in a stage-wise fashion, where each stage aims to improve upon the predictions of the previous stage. The model building process starts with a base model, often a decision tree. Gradient boosting builds upon insights from each previous model's output to progressively refine the overall ensemble's predictions. Gradient boosting is a powerful technique that can be used for both classification and regression tasks.

An illustrative machine learning algorithm may include transformers. Transformers are a deep learning architecture often used for natural language processing (NLP). Transformers are based on the concept of self-attention, which allows the model to attend to different parts of the input sequence and learn long-range dependencies. Transformers have achieved remarkable results on a variety of NLP tasks, such as machine translation, text summarization, and question answering.

The AI model may be configured to receive the voice input. The AI model may be configured to compute a second intent of the caller from the voice input. The AI model may compute the second intent by using one or more machine learning algorithms to derive an intent of the voice input.

Apparatus may include a middleware system. The middleware system may include one or more features of a computer system. The middleware system may be configured to formulate a response to the caller based on the first intent and the second intent. The middleware system may be configured to arbitrate between the first and second intents. The middleware system may be configured to limit risk of hallucination by comparing the second intent computed by the AI model to the first intent computed by the IVR system.

The computational options available to the IVR system may be more tightly controlled than the computational options available to the AI model. A human programmer may limit the computational options available to the IVR system. Using the IVR system to supervise output of the AI model may provide a more reliable system that is suitable for use in mission-critical healthcare applications.

The middleware system may be configured to throttle a level of regularization applied to the AI model. The middleware system may determine a level of regularization or regularization technique to apply to the AI model. The middleware system may make a regularization decision based on a confidence measure associated with the first intent.

The middleware system may compute a confidence measure for an intent generated by the IVR system or the AI model. The computed confidence measure may include a confidence level. The confidence level may represent a probability that an intent is an accurate interpretation of the voice input provided by the caller. The confidence level is typically expressed as a percentage. For instance, a 95% confidence level indicates that if the same voice input was received multiple times, the IVR system or AI model would generate the “true” intent 95% of the time. For a voice input, a “true” intent may refer to how a live human agent would have understood the caller's voice input.

The computed confidence measure may include a confidence interval. The confidence interval refers to generated intent that likely contains the “true” intent of the caller's voice input. For example, a 95% confidence interval means the IVR system or AI model has a 95% chance of generating an intent that includes the caller's “true” intent.

Regularization refers to a set of techniques used by machine learning algorithms to prevent an overfitting problem. Typically, an AI model relies on one or more machine learning algorithms to make a prediction. The AI model is provided a training dataset and programmed to learn and detect underlying patterns in provided data and use those patterns to make accurate predictions on new, unseen datasets. Overfitting occurs when an AI model memorizes the training data too well. The AI model behaves like a student who can only recite memorized answers but can't solve new problems that require applying concepts. Overfitting leads to poor performance on unseen data.

Regularization techniques act like training wheels for the AI model. Regularization adds constraints or penalties that prevent the AI model from becoming overly complex and fixated on every tiny detail in the training dataset. Regularization encourages the AI model to focus on capturing the general patterns that are more likely to generalize well to new datasets. Regularization may simplify the model by penalizing AI models that develop too many parameters or features. This discourages the AI model from becoming overly complex and fitting to random noise in datasets. Regularization may add a penalty term to the AI model. The AI model may then try to minimize the penalty term during training, which pushes the AI model towards simpler solutions that achieve good accuracy without overfitting. Regularization techniques may include data augmentation: This technique includes artificially creating new variations in the existing training datasets. By exposing the AI model to a wider range of examples, it can learn more generalizable patterns.

Regularized AI models perform better on unseen data because they haven't memorized the training data to a fault. Regularization also helps to stabilize an AI model's performance, making it less sensitive to small changes in the training data. Regularization helps the AI model avoid becoming overly specific to the training data.

The middleware system may be configured to pass the response it formulates to the IVR system. The IVR system may be configured to generate audio output based on the response. The IVR system may utilize conversational AI to generate the response provided to the caller. Conversational AI may refer to a computer program that provides a conversational experience that mimics actual conversations with another human.

In a conversational AI system, the IVR system may use an AI model to understand the customer's intent and respond with relevant information. Conversational AI allows the IVR system to provide responses to caller questions quickly and efficiently without requiring the callers to navigate through multiple menu options as in a conventional IVR system. Conversational AI allows the IVR system to provide callers open-ended prompts and compute accurate intents of caller responses to those open-ended prompts.

The conversation AI system may allow callers to communicate with an IVR system naturally and using complete sentences. In a conversational AI system, the IVR system may respond to a caller using natural language speech. The natural language speech output provided by the IVR system may be pre-recorded or generated dynamically.

The conversation AI system may include speech-to-text (“STT”) and text-to-speech (“TTS”) technology. The IVR system may capture a voice input provided by a caller and use STT technology to convert the captured voice input into text. After the middleware system generates a response to the voice input, TTS technology may be used to convert a text-based response into speech output. SST and TTS technology may account for different languages, dialects, and regional accents. An AI-model coupled to STT and TTS technology allows an IVR system to engage in human-like conversations and generate lifelike speech that express emotion and vocal nuance.

The middleware system may be configured to increase the level of regularization based on a threshold confidence measure associated with the first intent. The middleware system may decrease the level of regularization when the first intent is above a threshold confidence measure.

The middleware system may be configured to instruct the AI model to compute the second intent before the IVR system computes the first intent. The middleware system may compute a confidence measure associated with the second intent. When the confidence measure associated with the second intent is below a threshold level, the middleware system may instruct the IVR system to compute the first intent.

The IVR system may be configured to compute an intent based on a fixed set of responses that have been reviewed by a human programmer. The human programmer may use the AI model to generate the fixed set of responses. However, the fixed set of responses may only be added to the IVR system after review by the human programmer. The intent generated by the IVR system may be immune from hallucinations. Computing two intents for a voice input by two different systems (IVR system and AI model) may reduce impact of any hallucinations generated by the AI model.

The middleware system may be configured to instruct the IVR system to compute the first intent. The middleware system may assess a confidence measure associated with the first intent. When the confidence measure associated with the first intent is below a threshold level, the middleware system may instruct the AI model to compute the second intent. By first computing the second intent, the middleware system may compare the AI generated second intent to a first intent computed by the IVR system based on a fixed set of responses that have been reviewed by a human programmer.

The AI model may be configured to recursively train itself using a plurality of first intents generated by the IVR system. For example, the AI model that includes gradient boosting may utilize the plurality of first intents to train a new decision tree. The first intents computed by the IVR system may be based on a fixed set of responses that have been reviewed by a human programmer. Using the first intents as training data may further prevent the AI model from generating a hallucinatory second intent.

In some embodiments, the AI model may be configured to generate a plurality of intents in response to a prompt presented to a caller. The IVR model may be configured to determine whether a voice input received from a caller matches one of the plurality of intents generated by the AI model. Thus, rather than the AI model itself deciding an intent of the caller, the IVR system may decide whether the intent generated by the AI engine is non-hallucinatory. Such oversight by the IVR system may further prevent the AI model from generating a hallucinatory intent.

The AI model may be configured to activate the IVR system and the middleware system in response to detecting a pre-defined trigger event. The pre-defined trigger event may be an inclement weather event at a target location. For example, if the AI model detects a snowstorm at a target location, the AI model may activate the IVR system to answer calls dialing the target location because it is likely that the target location will be understaffed.

The trigger event may be a time outside regular business hours such as nights, holidays, or weekends. The AI model may activate the IVR system to answer calls dialed during times when employees are typically not available to answer calls.

Apparatus may include a computer program. The computer program may include instructions that when executed on a processor on a computer system, cause the computer system to perform one or more functions or actions.

The computer system may include hardware and software. Hardware may include a Central Processing Unit (CPU). The CPU may be responsible for processing instructions and performing calculations. The CPU may control overall speed and performance of the computer system. The hardware may include Random Access Memory (RAM). RAM typically refers to temporary storage that holds data that the CPU is actively using. RAM allows the CPU to quickly access frequently used information.

Hardware may include a storage device which data persistently even when the computer system is powered off. Illustrative storage devices may include hard disk drives (HDDs) and solid-state drives (SSDs). SSDs typically offer faster data access compared to HDDs. Hardware may include a motherboard. The motherboard is a central circuit board that connects all the other hardware components and allows them to communicate with each other. For example, the motherboard may connect the CPU to a Graphics Processing Unit (GPU). GPUs, while traditionally used for video processing, are great at handling complex mathematical tasks. GPUS are increasingly important for accelerating demanding applications like video editing and machine learning algorithms.

Hardware may include input and output devices. Input devices allow human users to interact with the computer system. Illustrative input devices include keyboards, mice, touchscreens, webcams, and microphones. Output devices display information processed by the computer. Illustrative output devices include monitors, printers, and speakers.

The computer system may include software. Software may include a set of instructions that tell the hardware what to do and how to function. Software may include system software and application software.

System software manages a computer system's resources and provides a platform for running other programs. Examples include operating systems (like Windows, macOS, or Linux) and device drivers that control the operation of specific hardware components part of the computer system. Application Software allows users of the computer system to perform specific tasks. Examples of application software include web browsers for browsing the internet, word processors for creating documents, and video games for entertainment.

A computer program may include instructions that configure a computer system to answer a telephone call from a caller. The instructions may prompt the caller to request a service that requires integration with an electronic health record (“EHR”) system. The instructions may communicate with the EHR system and provide the requested service to the caller during the telephone call.

The instructions may prompt the caller using lifelike speech. Lifelike speech may include computer generated speech that expresses emotion and vocal nuance. Lifelike speech may mimic speech in a human-to-human conversation.

The computer program may generate the lifelike speech using an AI model that converts text into the lifelike speech. The computer program may prompt the caller using the lifelike speech. The computer program may convert the text into the lifelike speech by generating, based on a computed meaning of the text, natural sounding: intonation, resonance, voice and tone.

The computer program may prompt the caller in a first language. In response to detecting a voice input provided by the caller in a second language, the computer program may interact with the caller in a second language.

The computer program may receive a voice input from the caller and determine whether the voice input includes a unique speech signature associated with the caller. In response to detecting the unique speech signature in the caller's voice input, the computer program may locate a record corresponding to the caller and stored in the EHR system.

Conventional IVR systems and call centers use a time-consuming knowledge-based authentication process. Callers are asked to their social security number, date of birth, or mother's maiden name. The computer program may utilize an AI model to provide real-time caller voice-based authentication. The computer program may analyze a caller's voice characteristics and provide a real-time decision on a caller's identity.

The computer program analyzes the caller's speech characteristics such as rhythm, pitch, and tone to create a unique speech signature for a caller. When a voice input is subsequently captured from the caller, the computer program may automatically attempt to match the voice input to the stored unique speech signature. If the voice input matches the unique speech signature, the caller is authenticated. The caller does not say any specific phrases or words or otherwise interrupt a natural conversation flow.

The computer program may receive a service request from a caller. In response to receiving the service request, the computer program may automatically formulate an Application Programming Interface (“API”) call. The API call may be formatted in accordance with one or more requirements of an EHR system. The computer program may receive a response to the API call from the EHR system. The computer program may extract data from the response provided by the EHR system and present the extracted data to the caller. The computer program may reformat the data for consumption by the caller.

The computer program may present the extracted data to the caller during a telephone call using lifelike speech. The computer program may present the extracted data to the caller during a telephone call via text message. The computer program may present the extracted data to the caller during a telephone call using a first communication channel and a second communication channel. The first communication channel may present the extracted data to the caller as speech. The second communication channel may present the extracted data to the caller as a text message.

The computer program may facilitate bi-directional communication between an IVR system that answers the telephone call and the EHR system. The computer program may facilitate bi-directional communication between the IVR system and two or more EHR systems. The computer program may facilitate bi-directional communication between two or more IVR systems and the EHR system.

During a telephone call, the computer program may capture at least two data elements that uniquely identify the caller. The caller may provide the two data elements as one or more voice inputs. The caller may provide the data elements as natural language conversational speech. An illustrative data element may include a birthdate. An illustrative data element may include a telephone number. An illustrative data element may include a caller's first name. An illustrative data element may include a caller's last name.

Based on at least two data elements, the computer program may formulate an API call to search in the EHR system for a patient record corresponding to the caller. In response to locating the patient record, the computer program may provide a requested service to the caller using information stored in the patient record. In response to failing to locate the patient record, the computer program may formulate a second API call. The second API call may create, within the EHR system, a new patient record corresponding to the caller. The newly created patient record may include the two data elements captured from the caller.

The computer program may concurrently answer multiple telephone calls from multiple callers. The computer program may answer each telephone call after fewer than three rings. The computer program may be configured to limit a duration of the telephone call to 5 minutes or less. The computer program may be configured to provide the requested service to the caller in 5 minutes or less from a start of the telephone call.

Illustrative services that may be requested by a caller and provided by the computer system include booking a new appointment, cancelling an appointment or rescheduling an appointment. The computer program may customize the service for the caller. For example, based on contextual awareness the computer program may search for available appointment slots that conform to a target appointment template. The target appointment template may include a target date, time, provider or location. Based on the patient record extracted from the EHR system, the computer program may automatically greet the caller by name and offer the earliest available appointment that fits the target appointment template.

The computer program may customize the service for the caller by formulating an API call to the EHR system based on inputs captured from the caller during the telephone call, information extracted from the EHR system and optimization criteria set by a medical office that uses the EHR system.

Apparatus may include an automated system for scheduling an appointment during a phone call. The automated system may include an interactive voice response (“IVR”) system. The IVR system may be configured to answer a phone call received from a caller. The IVR system may be configured to concurrently answer phone calls received from a plurality of callers. The IVR system may be configured to begin interacting with each caller within a threshold time limit. The threshold time limit may be defined based on a number of rings before the IVR system answers and begins interacting with a caller.

The IVR system may be configured to receive a voice input from a caller. The voice input may be provided by the caller in natural language. The IVR system may be configured to accept the voice input in any of at least 30 languages. The IVR system may be configured to provide audio output in any of at least 30 languages. The IVR system may provide the audio output in lifelike speech.

The automated system may include a middleware system. The middleware system may be configured to schedule an appointment for the caller based on the voice input. The middleware system may be configured to schedule the appointment via an integration with an electronic health record (“EHR”) system. The middleware system may be configured to integrate with a plurality of EHR systems.

The IVR system may be configured to authenticate the caller using the voice input. The IVR system may be configured to analyze voice characteristics of a second voice input received from the caller. The voice characteristics may include rhythm, pitch, and tone, timbre, intensity, accent, pronunciation or cadence of voice input captured from the caller.

Based on the voice characteristics, the IVR system may generate a digital voiceprint corresponding to the caller. The IVR system may utilize the digital voiceprint to perform a real-time identify decision of the caller in response to receiving the first voice input.

The IVR system may be configured to extract information from the voice input received from the caller. The middleware system may be configured to use the extracted information to formulate a search for a patient record within the EHR system. For example, the extracted information may include the caller's name, birthdate, insurance information, address, phone number or desired provider. The middleware system may be configured to create a new patient record within the EHR system when the patient record is not located within the EHR system in response to the search.

The voice input received from the caller may be a first voice input. When the patient record is not located within the EHR system, the middleware system may trigger the IVR system to prompt the caller for a second or additional voice inputs. The middleware system may formulate additional searches for the patient record within the EHR system using information extracted from the second or additional voice inputs.

Based on the voice input captured by the IVR system, the middleware system may be configured to cancel an appointment for the caller in the EHR system. Based on the voice input captured by the IVR system, the middleware system may be configured to reschedule an appointment for the caller in the EHR system.

The middleware system may be configured to extract at least one available appointment slot from an EHR system. The IVR system may be configured to send a text message to the caller that includes details of the available appointment slot. The IVR system may be configured to present the caller with an audio description of the details of the available appointment slot. The caller may be presented with both the audio description and the text message that includes details of the available appointment slot.

The voice input received from the caller may include insurance information. The insurance information may be extracted from the voice input by the IVR system. The middleware may be configured to search for an available appointment slot with a service provider that is associated with the insurance information. The middleware may be configured to only present the caller with available appointment slots for providers that accept the caller's insurance.

The IVR system may extract a date and time from the voice input. The middleware system may be configured to search for an available appointment slot for the caller on or within a threshold temporal distance to the date and time. An illustrative threshold temporal distance may be within 36 hours of the date and time provided by the caller.

The middleware system may be configured to search for an available appointment slot for the caller based on a previous appointment scheduled by the caller. The middleware system may be configured to search for an available appointment slot for the caller based on a demographic characteristic of the caller. The demographic characteristic may be extracted from a patient record retrieved from the EHR system. The middleware system may be configured to search for an available appointment slot for the caller based on a plurality of appointments previously scheduled by other callers that each share one or more demographic characteristics in common with the current caller.

The middleware system may be configured to search for an available appointment slot for the caller that has a target duration. The middleware system may determine the target duration based on a demographic characteristic of the caller. The middleware system may determine the target duration based on a type of service requested by the caller. The type of service may be extracted by the IVR system from the voice input. The type of service may be determined based on a reason for scheduling the appointment provided by the caller. The caller may provide the reason in natural language. The IVR system may correlate the reason provided in natural language to one or more predefined service types. Illustrative service types may include cosmetic, medical, follow up care, new patient or emergency service.

The middleware system may be configured to allow overbooking when scheduling an appointment for the caller. The middleware system may be configured to determine whether to allow overbooking based on a demographic characteristic of the caller, a provider associated with the appointment, and/or a date and time of the appointment. For example, some providers may be comfortable when patients are overbooked. Other providers may feel overwhelmed when patients are overbooked. Based on the provider assigned to the appointment, the middleware system may determine whether to overbook an appointment slot.

The middleware system may be configured to present one or more available appointment slots to the caller. The middleware system may be configured to present an available appointment slot to the caller based on an attribute of a prior appointment scheduled by the caller.

The middleware system may decide which available appointment slots to present to the caller based on a set of filtering rules. The filtering rules are configured to achieve a target optimization in a schedule. An illustrative target optimization may include grouping scheduled appointments into predefined blocks of time. The filtering rules may be programmable from within a user interface associated with the middleware system. Illustrative filtering rules may be set for different caller profiles, office location, providers, times of day or combinations thereof. For example, the middleware system may be configured to present available appointment slots to the caller based on being programmed to fill a target block of time for a target provider at a target office location.

An AI model may dynamically set the filtering rules. The filtering rules may be dynamically set by the AI model based on real-time availability of appointment slots at an office location or with a specific provider. The filtering rules may be dynamically set by the AI model based on a caller's voice characteristic. The voice characteristic may be determined based on the voice input received by the IVR system. The filtering rules may be dynamically set by the AI model based on a reason provided by the caller for requesting the appointment. The filtering rules may be dynamically set by the AI model based on a characteristic of a provider selected by the caller. Provider characteristics may include overbooking rate, average number of patients seen per day, average appointment length.

The IVR system may be configured to accept the voice input as expressed by the caller in natural language. The voice input provided by the caller and received by the IVR system may include a birthdate. The IVR system may be configured to extract a birthdate of the caller from the voice input. The voice input may include one or more of a caller's: given name, last name, a birthdate, mailing address, desired provider, insurance carrier, desired appointment time (date, a day of the week and time of day).

The IVR system may be configured to convert the voice input into text. The middleware system may be configured to correct a spelling of the text using data from a reverse phone lookup. A number used by the caller to dial the IVR system may be used and the input for the reverse phone. For example, the voice input may include a name of the caller. The middleware system may be configured to correct the spelling of the name using the data obtained from the reverse phone lookup.

The IVR system is configured to extract a desired appointment date and time from the voice input. The IVR system is configured to extract a desired provider from the voice input. The middleware system may be configured to search for an available appointment slot that meets scheduling criteria expressed by the caller.

The IVR system may be configured to prompt the caller using an audio prompt expressed in natural language. An AI model may be configured to dynamically control content of the audio prompt. For example, the AI model may adjust the prompt based on the time of day or recent activity of the caller. The middleware system may be configured to adjust a characteristic of a voice output generated by the IVR system based on a characteristic of the voice input.

The middleware system may be configured to activate a target communication channel. The target communication channel may be used to transmit information to the caller during the phone call. The target communication channel may include a video call, a text message, an email message, an audio message, an automated bot and an online form.

The IVR system may be configured to initiate a primary communication channel during the phone call. The middleware system may be configured to trigger activation of at least one secondary communication channel during the phone call. The primary communication channel and the secondary communication channel may be active concurrently. For example, the caller may be presented with details associated with available appointment slots in lifelike speech and in a text message.

The AI model may be configured to determine, during the phone call, whether to transfer the caller to a live agent. The AI model may be configured to activate the target communication channel. The AI model may be configured to activate the target communication channel based on a content of information that will be transmitted to the caller using the target communication channel.

For example, if the AI model determines that it is more intuitive to consume the content in a text form, the AI model may send the caller a text message. If the AI model determines that it is more intuitive to consume the content verbally, the AI model may send the caller an audio message. The audio message may be generated and/or delivered by the IVR system. The AI model may be configured to dynamically control a characteristic of voice output generated by the IVR system.

The AI model may be configured to dynamically adjust functionality of the middleware system that is available to the caller during the phone call. For example, the AI model may restrict whether the middleware system allows the caller to utilize the IVR system for a target appointment related service. If the call has a history of no shows or frequent cancellations, the AI model may not allow the caller to cancel an appointment using the IVR system.

The AI model may be configured to dynamically generate an appointment template for the caller. The appointment template may include a date, time and provider. The AI model may submit the appointment template to the middleware system. The middleware system may search for an available appointment slot for the caller that fits the appointment template.

Apparatus and methods: improving workflows in a healthcare facility are provided. An illustrative healthcare facility may include three primary sections. The “front desk” is where office staff answers the phone, makes appointments, checks-in patients, helps patients complete forms (consents, questionnaires, insurance information). Patients may also provide payments for service at the front desk.

The “mid-office” includes exam rooms and is typically staffed by providers such as medical assistants, nurses, doctors, physician's assistants. The mid-office may receive updates from the front office on which patients have arrived and when they are ready to be examined. The “back-office” houses the administrative staff responsible for billing and submitting insurance claims.

The mid-office staff may use software to document the examination of the patient and update the patient's medical record. Documentation of a patient examination is called a “SOAP” note. The SOAP note is and is stored in an EHR system as part of the patient's record. A SOAP note includes four sections-subjective, objective, assessment and plan.

The subjective section includes a patient's perspective on their health concerns. It includes their chief complaint (the main reason for the visit), their medical history, current symptoms, and any other information they consider relevant. Providers typically obtain this information through direct questioning of the patient.

The objective section focuses on the providers findings based on a physical examination and any diagnostic tests performed. It includes vital signs (blood pressure, heart rate, temperature, respiration rate), physical exam findings (observations from looking, listening, feeling, etc.), and results of any lab tests, X-rays, or other diagnostic procedures.

The assessment section includes the providers interpretation of the information gathered in the subjective and objective sections. The provider analyzes the patient's symptoms, medical history, and test results to arrive at a diagnosis or a differential diagnosis (a list of possible diagnoses). This section also includes the provider's judgement about the severity of the condition and the prognosis (potential outcome).

The plan section outlines the treatment plan for the patient. It details the medications prescribed (if any), any recommended procedures or tests, referrals to specialists (if needed), patient education materials, and follow-up appointments.

Apparatus may include an electronic health record (“EHR”) system. An illustrative EHR system may electronically store medical information about a patient in a secure and centralized location. The EHR system may electronically store patient information as such demographics, medical history, allergies, medications, and immunizations. The EHR system may electronically store clinical data such as doctor's notes, test results, radiology images, diagnoses, and treatment plans. The EHR system may include tools to support care such as drug interaction checking, clinical decision support systems, and patient portals.

Apparatus may include a practice management system. An illustrative patient management system may include a software solution that helps manage the administrative side of patient care, streamlining workflows and improving efficiency. An illustrative patient management system may manage tasks like scheduling appointments, handling billing and insurance, and storing basic patient information. A patient management system may include communication tools for sending appointment reminders and lab results electronically and may offer features for secure patient-provider messaging.

Apparatus may include a kiosk. The kiosk may include one or more features of a computer system. The kiosk may be configured to access data of a patient stored in the EHR system and/or a practice management system. Based on the patent data, the kiosk may present target content to the patient. The kiosk may include a processor and a non-transitory memory storing computer executable instructions. The executable instructions, when executed by the processor may implement a function of the kiosk. The kiosk may formulate the target content based on the data of the patient stored in the EHR system and/or the practice management system.

The executable instructions, when executed by the processor may implement one or more AI models. The AI models may formulate the target content to present to the patient based on the data of the patient stored in the EHR system and/or the practice management system. The AI models may utilize one or more of the following computational techniques: linear regression, logistic regression, decision trees, support vector machines, naive bayes, K-nearest neighbors, K-means, random forests, dimensionality reduction, gradient boosting and adaptive boosting. The AI models may utilize one or more of the following computational techniques: supervised machine-learning, unsupervised learning machine-learning, semi-supervised machine-learning and reinforcement machine-learning.

The target content may include information about diagnoses, conditions, procedures, products, specials and/or services that are available in the healthcare facility. The target content may include videos, before/after pictures and frequently asked questions about the diagnoses, conditions, procedures, products, specials and/or services that are available in the healthcare facility. The procedures, products, specials and/or services may be purchased using the kiosk.

The target content may be formulated using one or more AI algorithms. The one or more AI models may be executed locally on the kiosk. The one or more AI models may be executed on a cloud-based computer server.

The target content may include discounts and/or specials for one or more of the procedures, products and/or services available in the healthcare facility. The target content may include an expected wait time until a provider is available to examine the patient. The target content may identify a staff member that will summon the patient into an exam room.

The target content may include a workflow that is presented to the patient. The workflow may be presented to the patient on the kiosk. The workflow may be presented to the patient on a mobile device of the patient. A first segment of the workflow may be presented on the kiosk and a second segment of the workflow may be presented on the mobile device. The workflow may be presented to the patient before the patient is examined by a provider. The workflow may be presented to the patient after the patient is examined by a provider. The workflow may be presented to the patient in an exam room. The workflow may be presented to the patient in an exam room before, during and after an examination of the patient by a provider.

The workflow may include a message for a provider. The kiosk may present an indicator that the message should be reviewed by the provider before examining the patient. The kiosk may authenticate the provider before the indicator is presented to the provider. The message may include a request from a guardian of the patient on how the provider should communicate with the patient.

The kiosk may detect a target motion or display of a target symbol. In response to detecting the target motion or display of the target symbol, the kiosk may set off an alarm or execute a special workflow. The alarm or the special workflow may indicate that staff at the healthcare facility are in physical danger.

The target content may include a workflow that is customizable in response to input received from the patient, a family member, a guardian, a friend, or a staff member at the healthcare facility. The target content may include a workflow that is customized for the patient based on the data of the patient stored in the EHR system and/or the practice management system. The workflow may be customizable by allowing the patient or the staff member to bypass an input field included in the workflow, submit a change to the workflow or change a sequential order for completing the workflow.

The kiosk may include a camera. The kiosk may perform biometric authentication of the patient based on an image, a video or other information captured by the camera. The kiosk may detect a target area based on an image, a video or other information captured by the camera. The target area may be on a body of the patient. The kiosk may identify the target area based on detecting an object or a boundary within the image, the video or other information captured by the camera. The object may include a target marker, a target shape or a target symbol.

The camera may be capable of capturing a facial image that is suitable for performing biometric facial recognition. The camera may be capable of capturing an eye image that is suitable for performing biometric iris or retina recognition. The camera may be capable of capturing a fingerprint image that is suitable for performing biometric fingerprint recognition. The camera may be capable of capturing a document image that is capable of being converted into machine-readable text.

The kiosk may extract the information from an image. The image may be captured by the camera. The image may be stored on the kiosk or otherwise accessible to the kiosk from a remote location. The image may be a digital photo of an insurance card. The kiosk may extract information from the image. Based on the extracted information, the kiosk may determine insurance eligibility and insurance attributes for the patient. The insurance attributes may include a name or description of an insurance plan, an insurance carrier, an insurance plan number, a copayment amount, a deductible amount, a coinsurance amount, services covered by the insurance plan and/or out-of-network benefits associated with the insurance plan. The insurance eligibility may include a start date of insurance coverage, an end date of insurance coverage and/or whether the patient is current on premium payments for the insurance coverage.

The kiosk may include a digital wand or pen. The kiosk may identify a target area based on movement of the digital wand or pen. The movement of the digital wand or pen may include virtually outlining the target area. The camera may be configured to automatically capture one or more photos of the target area. The kiosk may adjust one or more image capture settings of the camera and then capture one or more photos of the target area. The kiosk may determine that one or more photos need to be retaken. The kiosk may automatically retake one or more photos of the target area.

The kiosk may include a digital signature pad. The kiosk may convert hand-written text captured by the digital signature pad into machine-typed text. The kiosk may include a scanner. The kiosk may extract information from an image captured by the scanner. The kiosk may display an image captured by the scanner and mark a field within the image that includes extractable information. The kiosk may allow resizing of the mark applied to the field. The kiosk may allow entry of a custom description of the field. The kiosk may extract information from the field. The kiosk may associate the extracted information with a description of the extracted information.

The kiosk may allow changes to the extracted information. The kiosk may confirm the extracted information by querying and examining the data of the patient stored in the EHR system and/or the practice management system.

The kiosk may formulate sign language motions. The kiosk may present the sign language motions on a screen of the kiosk. The kiosk may present the target content using voice. The kiosk may present the target content using voice interaction that mimics a sound, expressions, personality and/or mannerism of a provider that that is scheduled to examine the patient. The kiosk may present the target content using an avatar exhibiting a likeness and/or a mannerism of a provider that is scheduled to examine the patient.

The kiosk may be configured to interact with the patient using voice. The kiosk may be configured to receive voice input from the patient. The kiosk may be configured to receive voice input from the caller in natural language. The kiosk may be configured to receive voice input from the caller in any one or at least 30 languages. The kiosk may generate and provide lifelike voice to the caller as output. The kiosk may be configured to receive touch input from the patient.

The kiosk may be configured to interact with a scheduling system to book an appointment for the patient. The kiosk may be configured to book the appointment based on appointment history data associated with the patient. The appointment history may include a number of times the patient has not showed up to previous appointments, a number of times the patient has rescheduled previous appointments, a number times the patient has cancelled the previous appointments, a number of times the patient has been late to the previous appointments, an amount of time the patient has been late to the previous appointments, a number of times the patient has been early to the previous appointments, an amount of time the patient has been early to the previous appointments, a number of times the patient has arrived on-time to the previous appointments, a total number of appointments scheduled on behalf of the patient, nature of services (e.g., elective or medically needed) associated with the appointment, monetary revenue expected to be generated by the appointment, patient behavior at the previous appointments and/or total duration of each of the previous appointments relative to scheduled time for each of the previous appointments and duration of appointments schedule for other patients. Each factor included in the appointment history may not be assigned an equal weight when the kiosk determines how to book the appointment.

The kiosk may generate a reminder about an upcoming appointment. The kiosk may transmit the reminder electronically to the patient (e.g., email or text message). The kiosk may generate the reminder based on data of the patient stored in the EHR system and/or the practice management system. The kiosk may generate the reminder based on appointment history data associated with the patient. The appointment history data may include: a number of times the patient has not showed up to previous appointments, a number of times the patient has rescheduled previous appointments, a number times the patient has cancelled the previous appointments, a number of times the patient has been late to the previous appointments, an amount of time the patient has been late to the previous appointments, a number of times the patient has been early to the previous appointments, an amount of time the patient has been early to the previous appointments, a number of times the patient has arrived on-time to the previous appointments, a total number of appointments scheduled on behalf of the patient, a nature of services (e.g., elective or medically needed) associated with the appointment a monetary revenue expected to be generated by the appointment patient behavior at the previous appointments and/or total duration of each of the previous appointments relative to scheduled time for each of the previous appointments and duration of appointments schedule for other patients.

The kiosk may be configured to interact with a timekeeper system. The kiosk may be configured to track attendance of a staff member working at the facility. In response to detecting a change in a regular schedule of a staff member, the kiosk may prompt the staff member to provide an explanation for the change. The kiosk may be configured to receive the explanation in audio, text or video format.

The kiosk may transmit the explanation to a target individual. The target individual may be defined by the staff member, a default rule and/or a supervisor of the staff member. The kiosk may prompt the staff member to provide an explanation for the change in their regular schedule based on: number of times the staff member has not showed up for work, a number of schedule changes requested by the staff member, a number of times the staff member has arrived late to work, an amount of time the staff member has been late to work, a number of times the staff member has arrived early to work, a number of times the staff member has arrived on-time to work, an amount of time the staff member has arrived early to work, a total number of days and/or hours the staff member has worked, a nature of service provided by the staff member, a value of services provided by the staff member and/or behavior (such as ratings of disposition, insubordination, argumentativeness, number of human resource write ups, helpfulness, creativity, proactiveness, leadership) of the staff member.

In response to detecting a change in a regular work schedule of a staff member, the kiosk may notify a supervisor of the staff member. In response to detecting a change in the regular work schedule of a staff member, the kiosk may provide target content to the staff member. The target content may include a message that is configured to help the staff member return to the regular work schedule. For example, the message may include an offer to assist the staff member with a personal challenge, an offer for the staff member to change their working days or hours, an encouraging message, scheduling a meeting with a supervisor and/or any other suitable message that is structured to maintain the staff member's regular work schedule.

In response to detecting unexpected activity at a facility, the kiosk may activate a camera and microphone to record the unexpected activity.

The kiosk may be configured to download, from the EHR system and/or the practice management system, a local copy of patient data. In response to detecting no or slow connectivity to the EHR system and/or the practice management system, the kiosk may provide output and capture input based on the local copy of patient data stored on the kiosk. The no or slow connectivity is due to an internet or power outage or slowdown.

In response to detecting restoration or increased speed of the connectivity to the EHR system and/or the practice management system, the kiosk may sync the local copy of the patient data with the EHR system and/or the practice management system. The kiosk may include a battery that allows the kiosk to operate for at least 5 hours without being connected to any other power supply.

A method for improving efficiency of a workflow in a healthcare setting is provided. The method may include executing computer executable instructions on a processor that configure a computer system to perform one or more tasks. For example, the method may include biometrically authenticating a patient. The method may include prompting the patient to scan a driver's license or other form of identification. The method may include extracting data from the driver's license or other forms of identification. Based on the data extracted from the driver's license or other form of identification, the methods may include confirming a name, an address and a date of birth of the patient.

Methods may include prompting the patient to scan an insurance card or other proof of insurance coverage. Methods may include extracting data from the insurance card or other proof of insurance coverage. Based on the data extracted from the insurance card or other proof of insurance coverage, methods may include determining insurance eligibility and insurance attributes for the patient. Based on the data extracted from the insurance card or other proof of insurance coverage, methods may include prompting the patient for a payment and presenting a target workflow to the patient.

Methods may include presenting the patient with information about procedures, products and/or services available in the healthcare facility. Methods may include presenting the patient with the information about procedures, products and/or services based on data stored in an EHR system and/or practice management system and associated with the patient.

Methods for improving workflow in a facility are provided. Methods may include presenting to a consumer information about products, services, procedures, specials available in a facility. Illustrative facilities may include one or more of: a nursing home, a medical office, an outpatient surgery center, an urgent care center, a hospital, an assisted living facility, a skilled nursing facility and/or any other healthcare facility.

Methods may include generating the consumer information presented to the consumer based on a need or goal of the facility. Methods may include generating the information presented to the consumer based on a need or goal of the consumer. Methods may include generating the information presented to the consumer based on a need of the consumer and the facility.

The kiosk may be positioned on a table, a stand or wall mounted. The kiosk may snap into a stand with wheels, which will allow the kiosk to be moved from room to room within an office. The kiosk may be mounted on a mechanical arm or on a wall or ceiling track. The mechanical arm or track may be motorized and allow the base unit to move between different positions based on captured input (e.g., camera, motion).

The kiosk may include a docking station. The docking station may enable connections to multiple peripheral devices and other accessories. Illustrative peripheral devices may include a keyboard, mouse, card scanner, barcode scanners, fingerprint readers, payment terminals, microphone, camera, printer, speakers, storage devices, near-field communication (“NFC”) readers/writers, routers and modems. Peripheral devices may include devices for measuring vital signs. Illustrative peripheral devices for measuring vital signs may include a pulse oximeter, a blood pressure monitor, a thermometer and an electrocardiogram (ECG) monitor.

The kiosk may integrate with a printer (to print receipts, upcoming or past appointments, referrals, prescriptions, name of items/products recommended by provider or other information provided to patient). The kiosk may integrate a patient's mobile device and provide information to the mobile device such as by adding a new appointment to a patient's calendar application, task manager app or to a mobile wallet.

An illustrative kiosk may include a screen. The screen may be between 10 inches and 36 inches and capable of displaying high resolution images (e.g., medical grade X-rays). The screen may be touch sensitive. An orientation of the screen may be mechanically changed to/from portrait or landscape. The screen may be moveable up/down/left/right, 360 degrees in either direction and/or move up/down. The screen may be mounted on a socket and ball joint that allows the screen to be positioned in a desired location.

The kiosk may include built-in speakers and microphones for voice interaction. The kiosk may include a camera. The camera may be capable of capturing images suitable for biometric face recognition. The camera may capture 3D images. The camera may be capable of capturing medical grade images (e.g., dermatological skin conditions). A medical grade image may refer to a digital image captured using medical imaging equipment that meets specific criteria for accuracy, clarity, and consistency. Such images are usable by medical professionals for diagnosing and treating patients.

Key characteristics of medical grade images may include high resolution and detail. Medical grade images are typically of exceptionally high resolution to allow providers to see even the finest details of anatomy and potential abnormalities. This enables providers to make accurate diagnoses and treatment decisions. Medical grade image may be of consistent quality. Unlike regular photos, medical grade images are typically consistent across different scans of the same patient or even between scans captured by different machines. This consistency ensures reliable comparisons over time or across facilities.

Medical grade images provide accurate representation. Medical grade images represent the patient's anatomy as faithfully as possible, with minimal distortion or artifacts (image imperfections) that could hinder interpretation. Medical grade image may be DICOM compliant. Often, medical grade images adhere to a specific format DICOM called (Digital Imaging and Communications in Medicine). This standard ensures compatibility across different medical imaging devices and software programs, facilitating image sharing and analysis within healthcare systems.

Equipment used to capture medical grade images may be subject to rigorous calibration and Quality Control requirements. Medical imaging equipment may undergo regular calibration and quality control checks to guarantee the accuracy and consistency of the images produced.

Illustrative medical grade images may be used for X-ray imaging (examining bones and fractures), CT scans (detailed views of organs, blood vessels, and tissues), MRI scans (examining soft tissues, the brain, and the spinal cord) and Ultrasound imaging (examining fetuses and internal organs).

The kiosk may include specialized cameras for capturing and documenting areas of concern on the patient's body. Examples of such cameras include the Casio DZ-S50 Dermoscope and the Casio DZ-D100 Dermocamera. Such cameras may detect and document skin damage (such as sun damage caused by exposure to UV light), skin cancer or other medical conditions.

The kiosk may include a device for storing electronic data. For example, the kiosk may include a 1 TB HDD or SSD. The kiosk may include a battery backup. The battery backup may power kiosk in the event of a power outage. An exemplary battery backup may power the kiosk for 10 or more hours. The kiosk may include wireless communication capabilities. The kiosk may include NFC (e.g., NFC Tag 2.1), Bluetooth (e.g., version 5.3) and Wi-Fi (e.g., version 6E or 7) connectivity or any other suitable wireless protocol or technology.

The kiosk may include universal serial bus (“USB”) ports. For example, the kiosk may include 4 USB ports. The docking station may provide additional USB ports. The kiosk may include a network hardware adapter. The kiosk may include a wireless network adapter. The kiosk may include an Ethernet port for connecting to a wired network. The kiosk may include one or more High-Definition Multimedia Interface (“HDMI”) ports. An HDMI port may be capable of communicating audio and video signals. The kiosk may include one or more USB-C ports that are suitable for communicating data, audio and video signals.

The kiosk may include one or more biometric sensors. Illustrative biometric sensors may include a fingerprint scanner, iris scanner, retina scanner, facial recognition system, voice recognition system and/or any other sensors.

The kiosk may include an optical image scanner. The optical image scanner may be capable of capturing a single side of a document or card. The optical image scanner may be capable of duplex scanning (capturing both sides of a card at same time). The optical scanner may be configured to read and extract information from a driver's license or other government issued identification cards. The optical scanner may be configured to read and extract information from an insurance card. An Illustrative scanning area may be 4×5 inches or larger. The optical scanner may capture any suitable image in any suitable format. The optical scanner may also support the reading of barcodes, QR codes or other machine-readable codes. The kiosk may include optical character recognition (“OCR”) software for extracting data from a scanned image. Information extracted from a captured image may be automatically added to the appropriate fields of the patient's EHR record. For example, the scanner may be capable of extracting information from cards that conform to ISO7811 or the American Association of Motor Vehicle Administrators (“AAMVA”) for driver's licenses and identification cards.

The kiosk may include a digital signature pad. The kiosk may include a digital pen for providing input that can capture a signature area on the digital signature pad. An illustrative signature area may be 3.5 in.×2 in. inches or larger. Inputs captured by the digital signature pad may be automatically added to an appropriate field of the patient's EHR record. The digital signature pad may also function as fingerprint reader.

The digital signature pad may be used to turn on or activate a kiosk. For example, the digital signature pad may be heat sensing and/or detect a pre-defined pattern drawn on the signature pad. The pattern, when detected, may unlock or lock public access to the kiosk.

The digital signature pad included in the kiosk may be connected to digital signature capture systems. The patient may sign to authorize waivers such as release of information protected by the Health Insurance Portability and Accountability Act (“HIPPA”) or to authorize future payments using a stored credit card. The kiosk may use an NFC chip, to facilitate reading of payment card information, identification cards or other radio-frequency identification (“RFID”) tags. Products sold in an office may include an RFID tag to track inventory and may be scanned by the kiosk's NFC reader during a checkout workflow.

The kiosk screen may be configured to automatically dim or turn off after a certain amount of time. Turning off the screen may reduce risk of exposure of confidential information displayed on the screen. Confidential information may be presented on the screen in a manner that is only visible to the current kiosk user. Confidential information may be presented on the screen such that it is not visible to others, even if they are standing behind the current kiosk users. For example, the kiosk screen may present information in 3D. The current kiosk user may need to wear specialized viewing equipment, such as a headset or glasses to clearly see the information presented on the kiosk screen. Without the specialized viewing equipment, confidential information presented on the kiosk screen may be obscured to others.

The kiosk may include a privacy “screen blocker” that prevents bystanders from observing patient input or information presented on the kiosk screen to the patient.

The kiosk may include a digital wand. The digital wand may be the same device as the digital pen described above. The digital wand may be used to virtually outline a target area. The target area may be on a patient's body. The digital wand may virtually outline the target area with or without contacting the patient's body. The patient may use the digital wand to mark a target area of concern for the provider to examine.

Movement of the digital wand through space may be tracked by the kiosk. The digital wand may include various sensors to enable the kiosk to track its motion. The digital wand may include an accelerometer which measures the wand's acceleration in different directions (X, Y, Z axes). The accelerometer may allow the kiosk to detect wand movements such as tilting, shaking, and swiping. The digital wand may include a gyroscope for measuring the wand's rotational motion around the X, Y, and Z axes. The gyroscope can detect more nuanced movements like rolling, pitching, and yawing, allowing for finer control.

The digital wand may include a magnetometer which measures the Earth's magnetic field and is used to determine the wand's orientation in space. The magnetometer can be used to track absolute movements or for applications where the wand's direction is important. Magnetometers are sometimes used in conjunction with accelerometers and gyroscopes for enhanced spatial awareness.

The kiosk and/or wand may include a camera and depth sensors. The camera and associated depth sensors may be used to track the wand's position and orientation in 3D space. Some cameras may include built-in depth sensors, such as Time-of-Flight (“TOF”) sensors which can provide more accurate distance information. ToF sensors allow for highly precise tracking and enable features like gesture recognition for interacting with virtual objects. The wand may include direct ToF (“dToF”) and/or indirect ToF (“iToF”). dToFs directly measure the time difference between emitting a light pulse and receiving reflection of the light pulse from an object in front of the sensor. An iToF is configured to modulate the light wave and measures the phase shift between the emitted and received signals to determine the distance.

The wand may include Infrared (“IR”) sensors which can be used to detect the wand's position relative to the kiosk or other devices that include IR emitters. This can be useful for applications where the wand needs to be tracked within a specific area. The wand may include touch sensors to detect touch gestures on the wand's surface. Touch sensors may provide additional input for controlling the wand's functions.

Based on the tracked motion of the digital wand, a camera of the kiosk may focus on the outlined target area (e.g., on patient's body) during an office or telemedicine visit. The camera may automatically capture an image of the target area or highlight the target area on a general body image. The kiosk may automatically determine a focus, orientation or other settings for capturing a medical grade image. The kiosk may detect whether a captured image is blurry or out of focus and prompt for automatically retake the image.

The kiosk may detect a target motion of the digital wand. The target motion may be an instruction for the kiosk to capture the image or the type of image to capture (e.g., a close-up image, a zoomed-out image, use or not use flash). For example, the kiosk may detect that the wand has been moved in a check motion or “x.” This target motion may indicate that the kiosk should proceed to capture the area virtually outlined by the wand. The kiosk may provide voice instructions to a patient on how to position themselves so that the medical grade image can be successfully captured. The kiosk may instruct the patient to stay still or otherwise indicate that the image is about to be captured.

The kiosk may include functionality that allows a provider to outline a target area on the patient's body that should be captured by the camera. For example, the provider may hold an arrow or other object that will automatically be recognized by a camera of the kiosk. The patient or doctor may use their finger to point to the target area or position markers around the target area. Upon detection of the automatically recognized object, the kiosk may focus the camera on a target area (e.g., tip of arrowhead) and capture an image of that location. For example, the kiosk may automatically detect the finger, arrowhead or makers and focus the camera on the target area.

The kiosk may project a visible line on a floor where the patient should stand to capture a clear photo A provider may draw marks on the patient's skin. The ink may include fluorescence or metallic particles that are detectable by the camera or other sensor in the kiosk. The kiosk may then compute the location of the target area to photograph. The marks may be detected visually by the camera. Based on the marks or positioned objects, the kiosk may utilize location determination processes (e.g., triangulation) to focus on a target area.

In other embodiments the Kiosk may detect and track movement of the digital wand. A provider may move the digital wand (e.g., draw a virtual circle around an area of the patient's body). Based on the detected and tracked movement of the digital wand, the kiosk may focus its camera on the target area and capture an image of the area demarcated by movement of the digital wand. The kiosk may instruct its camera to take one, two or more medical grade photos of a target area. For example, the Kiosk may direct capture of a first photo of the target area, a second photo of the target area and neighboring areas (which may show a contrast between the target area and other parts of the patient's body) and third photo that is a close-up view of the target area. When photographing a rash on the patient's skin, a provider may need to capture a broader area (so that the photographed target area that includes the rash can be compared to neighboring unaffected skin).

When capturing a photo or a mole, the kiosk camera may need to adjust one or more image capture settings such as zoom-in to capture finer details such as contours and edges of the mole. Based on information stored in a patient medical record, the camera may auto adjust its image capture settings such as flash, continuous auto-focus, zero-shutter lag, frame capture rate, pan or zoom. For example, the kiosk may be programmed to adjust camera properties based on the patient's chief complaint, updated information received from the provider, patient's past diagnoses or demographic data.

The kiosk may utilize object recognition, to detect the presence of other objects in an exam room (such a chair or exam table) or other environmental conditions such as ambient light conditions. The kiosk may compute an ideal resolution, lighting, zoom or other photograph setting based on the detected size of the surrounding objects or other environmental conditions. The kiosk may be programmed to automatically take a series of views needed for clinical diagnosis (one panoramic view that shows a target location relative to other body parts or landmarks and a second view that shows a close up of the target location).

The kiosk may identify an area of the patient's body. For example, the kiosk may identify a part of the patient's body captured in an image (e.g., arm, leg, head) and automatically add the photo to the patient's medical record. The kiosk may indicate on an anatomical diagram a specific body part that the image is associated with. The kiosk may superimpose previous pictures of a target location so that changes to a target area may be viewed and previously documented conditions removed or added. The kiosk may display previous pictures of a target location side-by-side so that changes to a target area may be viewed concurrently.

The kiosk may include a payment terminal. The payment terminal may include a reader configured to capture information from a payment instrument. Illustrative payment instruments may include credit cards, debit cards and digital wallets. The payment terminal may support reading of magnetic strips. The payment terminal may support reading NFC chips or other contactless communication protocols for capturing information from a payment instrument.

The kiosk may include an IVR system. The IVR system may capture and interpret voice inputs. The IVR system may generate voice outputs. For example, the IVR system of the kiosk may provide a voice response to patient that capture of credit card information was successful or explain why payment information was not captured.

The kiosk may include support for headphones or a microphone. The headphones or microphone may include noise cancellation technology. Noise cancellation technology aims to reduce unwanted background noise, creating a quieter and more immersive listening experience. The headphones or microphone may be connected to the kiosk via a wired link. The headphones or microphone may be connected to the kiosk via a wireless link, such as Bluetooth or any other wireless protocols.

The kiosk may include a power supply. An illustrative power supply may include a 100-110V power supply. The kiosk may include a microprocessor such as Intel Atom processors x7000E Series, Intel Processor N-series and Intel Core i3 N-series or any other suitable processors. The kiosk may include transitory and non-transitory memory. The kiosk may include an operating system such as iOS, Windows, Linux or Ubuntu.

The kiosk may plug directly into a docking station. The docking station may utilize the kiosk's power supply. The docking station may include ports (such as USB ports wireless adapters) for adding peripheral devices (e.g., measuring vital signs, additional cameras, biometric sensors). The docking station may include smart connectors that allow additional docking stations to be stacked. Such smart connectors may include magnetic connectors, pogo pins and other modular connectors.

The kiosk may be configured to be affixed and supported by a stand. An illustrative stand may include a 4-foot plastic or base metal stand that will support the kiosk. A base of the kiosk may snap into the top of the stand to securely hold the kiosk in place. The stand may include wheels and locking casters so that the stand may be positioned in a fixed position.

The kiosk and its IVR system may include support for conversing in multiple (e.g., 30+) languages such as English, Spanish, German, Chinese, Korean, French, Arabic and Hebrew. Utilizing its IVR system, the kiosk may interact with patients using speech and voice recognition. The IVR system may provide support for the kiosk to perform speech to text and text to speech dictation/transcription. The kiosk may use AI algorithms to interpret and compute intents for voice inputs provided by patients using natural, conversational language.

The kiosk may use AI algorithms to generate lifelike voices that mimic the voice, tone, expressions of a provider or other office staff member. The kiosk may attempt to derive a spoken pronunciation of a patient's name or any other word. The kiosk may record a desired pronunciation of a patient's name. The kiosk may transfer the desired pronunciation to the front desk or medical assistant, doctor or any office staff member. The kiosk may “mimic” patient's desired pronunciation as accurately as possible (e.g., when greeting the patient in the exam room or at a subsequent visit).

Mimicking the voice attributes of a provider may enhance credibility of the kiosk when communicating with a patient. The kiosk may use the voice of a famous cartoon character or other celebrity (e.g., President of the United States). A famous character or celebrity may be displayed on a screen of the kiosk showing a patient how to apply/take medication or follow other instructions. Pictures of the character or celebrity may be given to the patient as a reward for achieving a target level of compliance with instructions presented by the kiosk.

The kiosk may be configured to automatically detect a human face present within a threshold distance of the kiosk camera. In response to detecting the human face, the kiosk may automatically activate and greet the detected patient. The kiosk may authenticate patients using biometrics (such as facial unique speech signature) or prompt for a recognition or a conventional username/password.

The kiosk may include a middleware system. The kiosk may utilize the middleware system to query an EHR system. Based on responses from the EHR system, the kiosk may automatically recognize, greet and interact with patients. The kiosk may identify pre-existing patients. The kiosk may identify new patients and register their biometrics so that they will be subsequently recognized by the kiosk. The kiosk may register new patients. The kiosk may utilize the middleware system to create a new patient record within the EHR system.

The IVR system associated with the kiosk may allow patients to request assistance by asking for “help” or submitting other voice commands to the kiosk. Patients can interact with the kiosk by asking verbally for an explanation of office procedures, medical procedures/services, billing, or submit voice inputs requesting any other suitable information.

Information displayed on a screen of the kiosk may be mirrored on the computer screen of the receptionist or other office staff. Office staff can see what is being presented to the patient on the kiosk and automatically input any information or provide guidance to the patient on how to navigate through a workflow presented on the kiosk. Office staff may adjust the presentation of information on the kiosk based on a request of the patient or office needs. For example, a patient may wish to decline signing a waiver or change the order of a workflow. An AI model associated with the kiosk may dynamically adjust information presented to the patient based on inputs or requests of the patient or office staff.

After the kiosk greets a patient, the patient may be given an option to use bidirectional voice (use of headphones or earbuds will provide additional privacy), touch or combination thereof to continue interacting with the kiosk. When presenting voice output to the patient, the kiosk may also display the spoken text on its screen. The patient may turn off kiosk's voice/speech capabilities and view presented questions and provide touch or voice inputs. Audio volume and other kiosk features can be controlled by voice or touch inputs.

As part of a patient registration/intake process, the kiosk's peripheral devices may measure and record vital signs such as temperature, blood pressure, heart rate and oxygen levels. The kiosk may be connected to peripheral devices such as a thermometer, blood pressure cuff, a scale and an oximeter. Office staff may be alerted if any vital signs are outside of an expected normal range. The kiosk may automatically prompt a patient to repeat capture of vital signs when initially captured values are outside the patient's normal range or outside a normal range for a population that shares the patient's demographic characteristics.

Users of the kiosk may be photographed, and full biometrics will be captured during the patient registration process. Photos and biometrics may be stored in an encrypted data log. The encrypted data log may be stored locally on the kiosk. The encrypted data log may be stored in a secure cloud computing environment.

Two or more kiosks may be networked or otherwise linked to each other. Multiple kiosks on a network may be synched with each other. The syncing may allow a patient to be recognized regardless of which kiosk in the facility is used. An ability to seamlessly interact with any kiosk on the network may make patient interactions with the kiosk feel more natural. The syncing may allow information captured by one kiosk on the network to be made available at another kiosk in the network. Two or more kiosks on a network may be physically at different locations.

Each kiosk deployed in a medical office, despite providing different functionality and supporting different workflows, may be capable of seamlessly transferring patient and associated captured information to another kiosk within the office or on a connected network. For example, after checking in patients at a first kiosk, the first kiosk may direct the patient to a second kiosk in the exam room which will also recognize and greet the patient by name.

A kiosk may interact with a patient using a specific persona. For example, some patients may desire a more serious persona and others may wish to interact with a more comical persona. The patient or office may specify which personas are available on a kiosk. The kiosk may be programmed to mimic the persona of a provider that is expected to examine the patient during an office visit.

The kiosk may provide support for telemedicine services. A middleware system may provide complete integration with and access to patient records via the EHR system. The middleware may interact with an EHR via API calls.

The kiosk may facilitate scanning and digitizing any documents or information such as paper referrals or identification cards carried by the patient. Identification cards may include driver's license, passport, social security card and Insurance cards. The kiosk may use OCR technology to extract information from a scanned document or card. The kiosk may be configured to automatically input the extracted information into appropriate fields in of a patient record stored in an EHR system.

An ability to automate updating of patient medical records may reduce human resource costs needed to check-in patients. An ability to automate updating of patient records may reduce data entry errors, and thereby improve medical care. The kiosk may scan a bar code or other machine-readable code on a patient's identification card (driver's license and insurance cards) and thereby automate capture the information on the card. Illustrative information that may be captured by OCR or scanning a machine-readable code may include: first/last name, date of Birth, address, gender, height, eye color, insurance carrier, insurance plan number, credit card number, credit card expiration date, CCV code and any other data printed on a card or document presented by the patient.

The kiosk may automatically check and confirm a patient's insurance eligibility, associated benefits and obligations associated with the patient's insurance plan. The kiosk may determine co-pay, co-insurance, deductible, covered services, and out-of-network benefits. A middleware system associated with the kiosk may submit information captured from a patient's insurance card to a clearinghouse for verification. Clearinghouses help providers increase reimbursement rates by verifying a patient's insurance coverage before an appointment and checking each claim before it's submitted to the insurance company for payment. The clearinghouse may validate a patient's insurance coverage, which includes verifying their insurance is active, coverage limits, and flags potential out-of-pocket expenses. After the patient has been examined by a provider, the clearinghouse may scrub claims for errors and omissions, and reformat data to meet the specific requirements of each payer.

Data captured by peripheral devices may be uploaded into a patient's record and, via the middleware system, stored in an EHR system. Information uploaded into the patient's record may include voice memos or text input. The kiosk may include native support for EHR system workflows or custom workflows developed for the kiosk. Illustrative workflows that may be executed by the kiosk are described below.

The kiosk may maintain a digital log of all activities conducted using the kiosk. The log may track who provided input to the kiosk. The log may identify who provided input to the kiosk using voice recognition or any other biometric features. The kiosk may record video logs of individual sessions. The kiosk may maintain a record of interactions with the kiosk, inputs captured by the kiosk, outputs provided by the kiosk and internal operations of the kiosk during the recorded interactions.

The kiosk may be capable of dispensing medication or pills. The kiosk may authenticate patients based on biometrics or a username/password. The kiosk may include a secure compartment for storing medication. The secure compartment may be refrigerated. Biometric authentication may include a combination of two or more biometric features (e.g., face+iris+fingerprint) for added security. The secure compartment may only open after a patient is authenticated.

The kiosk may dose the correct amount of medicine (e.g., number of pills) from a central secure storage compartment. The kiosk may bottle or otherwise package medication before dispensing it to the patient. The kiosk may limit access of an authenticated patient to a specific area of the secure compartment. Even an authenticated patient may only be granted access to the specific area that contains medication authorized for release to the authenticated patient.

The kiosk may use a middleware system to double-check data entered manually or otherwise stored in an EHR system. For example, the kiosk may use information extracted from patient documents, identification cards, mobile devices and/or credit cards to validate data included in a patent record stored in the EHR system. The kiosk may confirm manually entered information by checking remote databases or other information sources. For example, the kiosk may confirm a spelling of a patient's name based on information extracted from a patient's driver's license or data obtained from a reverse phone lookup.

The kiosk may be configured to communicate with third-party devices and services. For example, the kiosk may be configured to provide information (e.g., lab report, images or visit summary) to a referring doctor or insurance company.

The kiosk may display machine-readable code. Scanning of the displayed code by a patient's mobile device may transfer an in-process kiosk session to the patient's mobile device. The patient may then continue the workflow started on the kiosk using their mobile device. For example, after the transfer, the patient may use the camera on their mobile device to capture a fingerprint, iris image, facial image or retina scan or other biometric information to authenticate the patient. The mobile device may transmit the captured biometric information to the kiosk for authentication.

A patient may be able to begin inputting data and interacting on their mobile device and then finish the started workflow (e.g., check-in procedure or medical history) on the kiosk. For example, the kiosk may display a QR code that when scanned by the patient's mobile device, links the patient's mobile device directly to a kiosk for completing the started workflow.

In some embodiments, scanning the displayed machine-readable code may allow the kiosk to obtain information from a patient's mobile device (pictures a patient's concern/condition, voice messages, name, mobile phone number or other data). Data provided by the mobile device may be transferred to the kiosk directly from the mobile device using any suitable communication protocol (e.g., cellular, Bluetooth, Wi-Fi, wired connections). In other embodiments, the EHR system may push a machine-readable code to the patient's mobile device. The EHR system may communicate with the patient's mobile device via a middleware system. When the pushed code displayed on the patient's mobile device is scanned by the kiosk, the kiosk will initiate a personalized session or initiate transfer of information from the patient's mobile device to the kiosk. A personalized kiosk session may be initiated based on data provided to the kiosk by the mobile device or encoded in the machine-readable code.

To protect patient personal health information (“PHI”) the kiosk may be configured to perform a data wipe between patient sessions or otherwise periodically remove PHI from temporary storage locations on the kiosk. The data wipe may remove all PHI stored in transient memory or non-transitory memory of the kiosk. All data stored locally on the kiosk may be encrypted to prevent unauthorized access. The kiosk may employ security techniques such as BitLocker to prevent access information even if there is an unauthorized removal of memory or hard drives.

The kiosk may include an ultraviolet (“UV”) light generator that is capable of denaturing bacteria/viruses on a kiosk surface. The UV light generator may be oriented to disinfect kiosk surfaces that are frequently touched (e.g., keyboard, mouse, stand, peripheral devices, touch screen, fingerprint sensor) and periodically disinfect those surfaces. A UV disinfection system may be embedded in a sensor (such as a fingerprint reader) for denaturing viruses and bacteria present on the surface of the sensor.

Prior to the start of a workday, the kiosk may be configured to download patient records (from an EHR system) for patients that are expected to be seen in the office that day. Those downloaded patient records may be securely stored locally on the kiosk. The kiosk may then be capable of performing biometric authentication, machine learning algorithms or other functions locally, without requiring an internet connection or any other connection to a remote server or database. The kiosk may therefore continue operating based on local data despite loss of internet connectivity or a power outage.

Computer code necessary for operation of the kiosk may be stored locally on the kiosk. Such code may allow the kiosk to perform one or more EHR system functions. For example, the kiosk may be capable of biometrically authenticating patients, creating new patient records, recording notes, “charting” a patient despite loss of internet connectivity or power outage. The kiosk may upload any patient record created or updated while offline to the EHR system after internet connectivity or power is restored.

The kiosk may send messages (e.g., facsimile, text, WhatsApp, email, push notifications) to a patient's mobile device. Illustrative messages may inform a patient when they will be called into the exam room, which office staff member will summon them from the waiting room, which exam room will be used and when the provider is expected to examine them. Based on the status of other patients being seen by a provider, the kiosk may provide patients with HIPPA compliant updates on the provider's real-time availability. For example, the kiosk may detect that a provider needs to perform an emergency procedure on a first patient and may provide a second patient with an update that the provider will be delayed because of an emergency in connection with an earlier scheduled patient.

The kiosk may include a middleware system that facilitates integration with X-Ray, CT scan, MRI or other medical imaging systems. Images captured by other medical imaging systems may be displayed on the kiosk. An image displayed by the kiosk may be annotated (by provider or patient). Using a digital wand or pen, the provider or patient may draw or write (by hand) annotations on the displayed image.

Based on captured images of a patient's condition, an AI model associated with the kiosk may generate a diagnosis of a condition or potential treatment options. For example, the kiosk may detect sun damage or scars on an image of a target area of a patient's skin. Based on the measured damage to the skin or size of a scar, the AI model may recommend certain procedures or products.

The kiosk may integrate with timekeeper software or hardware. The kiosk may maintain, manage and secure timekeeper data. The kiosk may utilize facial recognition, fingerprints or other biometrics to authenticate employees, vendors or contractors that work in an office. The kiosk may use facial recognition or other biometrics to detect who is checking in or out. The kiosk may include software that determines whether the employee, contractor or vendor has arrived early, late or on time. The kiosk may prompt the employee, contractor or vendor to provide a text, audio or video message to a supervisor. For example, an employee may be prompted to leave a text, voice or video message explaining why they are leaving early or why they arrived late. The kiosk may update supervisors on why the employee is leaving early and advise how the supervisor can help the staff member maintain a regular, consistent schedule.

The kiosk may provide employees with messages about current employment policy such as that salary may not be paid for time worked prior to a previously scheduled start time or inform a vendor of a problem that needs to be fixed while the vendor is on site. If an employee is detected leaving early or comes late, they may be asked if everything is alright. The kiosk may use an AI model to determine how to respond to an employee or vendor. The kiosk may use the AI model to shift employee schedules and provide coverage if an office is expected to be understaffed or overstaffed. Employees may use the kiosk to check their hours and benefits. The kiosk may measure productivity of vendors and accuracy of invoices submitted by those vendors.

The kiosk may automatically detect when a patient enters the exam room. In response to detecting the patient, the kiosk may greet the patient by name. For example, the kiosk may detect a human face or heat or motion. The kiosk may position itself based on the detected location of the patient. For example, the screen of the kiosk may turn itself in the direction of the detected patient to “face” the patient as the patient enters the exam room. The kiosk may assume a particular profile when it detects and identifies a patient. Such a profile may include using specific facial expression, persona, language, accent, or playing specific music or introducing specific elective procedures or other information.

Based on an implemented persona, the kiosk may emit a smell associated with that person. For example, the kiosk may be capable of emitting one or smells (e.g., spraying from one of a plurality of embedded reservoirs). The kiosk may offer to purchase perfume or air fresheners, or lotion or creams associated with the emitted scent.

The kiosk may instruct the patient to make themselves comfortable and prepare to be examined by the doctor. The kiosk may determine necessary preparation (e.g., take off shoes and socks) based on patient record obtained from an EHR system. While the patient is waiting in the exam room, the kiosk may interact (e.g., using voice, touch or combination thereof) with the patient and present information about services available in the office (such as cosmetic or elective procedures), products sold in the office or promotions (e.g., gift cards, discounts, specials) available in the office. The kiosk may present videos, before and after pictures, FAQs and other information (e.g., pulled from medical journals or reliable internet sources) about relevant procedures and products.

The kiosk may track patient interest in presented products, procedures, services and promotions. The kiosk may create a note or record so that a provider or other office staff can see what the patient was interested in and follow up with the patient.

Based on information in the patient record or patient input (e.g., based on patient interaction with the kiosk), the kiosk may present videos, photos, before and after pictures and other information (e.g., pulled from medical journals or reliable internet sources) about a condition or complaint the patient is concerned about. The presented information may educate and calm the patient. Because the information is presented by the kiosk, the provider or other office staff may not need to spend as much time presenting or reviewing that information to the patient and instead may focus on treating the patient's medical concerns.

The kiosk may present information to a patient about a provider, such as credentials, experience, speaking engagements or authored journal articles. The kiosk may present information about other providers affiliated with an office.

The kiosk may respond to patient requests, such as a request to play desired music or adjust lighting in the exam room. For example, the patient may request that lights be dimmed, and window coverings closed. These customizations may calm the patient prior to examination by the provider.

The kiosk may include fall detection software that can detect when a patient may have fallen (e.g., fall off exam table or chair). Upon detection of a fall, the kiosk may automatically alert office staff.

During examination of the patient, the kiosk may display an anatomical diagram of a body part that the provider will examine. The kiosk may respond to voice prompts from the provider to rotate, pan, zoom or otherwise shift a position of the displayed image. Using the digital wand or pen, the provider may draw or otherwise mark on a screen of the kiosk, which area is clinically important and save the markings in the patient's record.

The kiosk may be instructed to turn its screen away or cover its sensors, while a patient is undressing. The kiosk may then wait for a voice instruction to face patient after the patient is wearing a gown. If the patient is expected to have a full body skin exam, the kiosk may automatically close the window coverings in the exam room to give the patient additional privacy. Such action by the kiosk may give the patient a sense of security, privacy and calm the patient.

The kiosk may be configured to operate offline and only connect to the internet periodically (e.g., between patients, at the end of a workday or when such connection is available). Such offline operation may mitigate risks that PHI or other HIPPA protecting information may be inadvertently disclosed. Offline operation may avoid office slowdowns due to internet or power interruptions. For example, at a designated time, all patient records for patients that have a confirmed appointment may be downloaded locally to the kiosk. All data entry and modifications may be capable of being performed offline. At the end of the day, all revised patient records will be uploaded to the EHR system.

A screen of the kiosk may present augmented reality (“AR”) content. The kiosk may include a projector that presents AR content in a large viewing format. For example, the Kiosk may present an enhanced, interactive version of a real-world environment achieved through digital visual elements, sounds, and other sensory stimuli via holographic technology. The kiosk may generate images of the patient showing simulated effects on an appearance of the patient of a medical, cosmetic or elective procedure. The kiosk may visually present information about a procedure or how a procedure is performed.

The kiosk may allow a patient to submit images of preexisting conditions. The kiosk may allow the patient to use their mobile device to transfer captured images to the kiosk. The kiosk may support any suitable data transfer protocol such as Bluetooth, Wi-Fi or any other suitable wired or wireless connection channel.

An IVR system included in the kiosk may communicate with the patient in any suitable language. The kiosk may include locally stored language packages or interface with cloud-based translation engines. The kiosk may allow the provider to speak in a first language and translate the provider's instructions into a second language when interacting with the patient. Likewise, the kiosk may allow the patient to speak in a first language and then translate the patient input into a second language when interacting with the doctor.

The kiosk may provide support for telemedicine services (e.g., for allowing a remote parent to join a child patient in the office). The kiosk may be mounted on a mechanical or robotic arm. The kiosk may be configured to detect a location of a patient (e.g., using directional voice detection or heat sensing) and automatically extend/retract and otherwise position itself relative to the patient based on a detected location or movement of the patient.

The kiosk may include a robotic arm that extends to the patient and affixes a medical device (e.g., oximeter) to the patient. The robotic arm may bandage a patient. The kiosk may integrate with other devices in an exam room. For example, the kiosk may adjust a height of an exam table based on a preference of a provider or prepare and open a biopsy bottle so that a specimen can be readily inserted by the provider in a sterile manner or otherwise provide access to other medical instruments or peripheral devices.

The kiosk may show on its screen where a patient should sit or what they should do in the exam room or any other pre-doctor engagement. The kiosk may project a light marker or other indicator that directs the patient to sit in a location or take a particular action (e.g., take off shoes or shirt).

The kiosk may generate notifications or alerts. Such alerts may be customized or may be autonomously generated by the kiosk. For example, the kiosk may alert office staff in response to detecting completion or non-completion of a patient registration process, abnormal vitals or if a patient prematurely leaves the exam room. The kiosk may provide patients with instructions on where to go or what to do next, next appointment confirmation, medications prescribed/reminder to pick up prescriptions.

The kiosk may register an office's daily open/close routine and be configured to detect anomalous behavior that is unexpected after hours. For example, the kiosk may detect a presence (or lack thereof) of a cleaning crew and may trigger an alert to law enforcement or office administrator if suspicious or otherwise unexpected activity is detected after hours or when no one is otherwise expected to be in the office.

The kiosk may utilize an AI model to review a patient's medical history and promote target procedures, products and/or services based on various medical, personal and demographic criteria. For example, the kiosk may utilize commercial AI engines such as those provided by OpenAI, L.L.C. located at 3180 18th St. San Francisco, CA 94110 (e.g., ChatGPT or InstructGPT) or those provided by Google LLC located at 1600 Amphitheatre Parkway Mountain View, California 94043 USA (e.g., Gemini). Illustrative criteria that may be utilized by such AI models may include Fitzpatrick skin type, size of pores, shape of face, and type of skin (e.g., dry, oily, scaly). The kiosk may interact with the patient based on various factors such as identity of the patient, location of the office, type of visit identity of the provider assigned to treat the patient and any other suitable factors. For example, the kiosk may assess sales tax for products sold in the office based on the office's location.

The kiosk may accept payments from a patient. The kiosk may recognize a patient's voice and accept voice authorizations of payments using previously stored payment information. The kiosk may capture biometric information from the patient and thereby authenticate the patient and authorize payments. In some embodiments, the kiosk may be configured to accept cash payments after authenticating a patient.

The kiosk may reduce and prevent insurance fraud by conducting multifactor biometric authentication of patients and office staff before billing an insurance company or authorizing a provider to treat a patient.

The Kiosk may be configured to integrate with ridesharing applications or other transportation systems and applications. For example, the kiosk may automate scheduling of a rideshare, taxi or paratransit service (such as the Access-a-Ride service provided by the Metropolitan Transportation Authority of 2 Broadway New York, New York 10004). The kiosk may automatically pre-fill the location of the medical office as a desired pickup location. The kiosk may automatically pre-fill a patient's home or use another default address as the drop-off location.

The kiosk may be configured to automate patient checkout. The kiosk may integrate with vending machines or components thereof. For example, the kiosk may include a robotic arm that can move a product ordered by a patient out of a storage location and provide it to the patient. In response to a request from a patient, the kiosk may send a message to the front desk or other office personnel to prepare a requested product for pickup by the patient. The kiosk may include dual screens. One screen may present information to a patient and the other screen may display the patient's progress to the front desk staff.

The kiosk may include a middleware system for integrating and interacting with appointment and scheduling systems. The kiosk may be used to book appointments using voice, text or other patient input. The kiosk may be configured to apply filtering rules that encourage patients to schedule appointments during specific time slots. The kiosk may offer incentives (e.g., discounts, transportation or promotional products) to encourage patients to schedule appointments during times/days that would otherwise be undesirable or are typically slow days at the office or other office/provider preferences.

The kiosk may use an AI model or other software to determine when to schedule an appointment. The kiosk may integrate into a call center or other automated scheduling software to allow patients to self-schedule an appointment and provide the patient with available or preferred time slots. The kiosk may obtain available appointment slots from an office's online scheduling system and present those options to a patient using voice interaction. The kiosk may receive voice or other inputs from the patient and schedule an appointment based on those patient inputs using the office's online scheduling system. A middleware system may facilitate interaction with the office's online scheduling system.

When scheduling appointments or reminders the kiosk may account for patient demographic information which may include current or past experiences, such as military or law enforcement or any other work history status. For example, patients having military or law enforcement experience may be more likely to arrive on time for an appointment and may need less reminders. When scheduling appointments or reminders the kiosk may account for a personality index or score (e.g., Myers Briggs) associated with the patient. A patient may self-identify themselves as being in a specific personality category and/or the kiosk may compute a personality category based on interaction with the patient. When scheduling appointments or reminders, extroverts may need more appointment reminders than introverts. When scheduling appointments or reminders the kiosk may account for a patient's past or current medical history. For example, a patient associated with a diagnosis (e.g., a life-threatening condition) or history of missing appointments may receive additional appointment reminders. When scheduling appointments or reminders the kiosk may account for a patient's culture or socioeconomic factors.

The kiosk may promote specials or other office products and procedures based on time of day/week/year and/or based on how much inventory is available or provider work schedules. The kiosk may be capable of taking or authorizing payment for a visit and prompt for reviews or testimonials. The kiosk may prompt and accept reviews/testimonials submitted via text, audio or video. The kiosk may prompt and receive suggestions or criticisms submitted via text, audio or video.

The kiosk will provide functionality that supports the following illustrative workflows:

TABLE 1
Illustrative Morning Office Workflow

| Step No. | Task Description |
|---|---|
| 1. | kiosk turns itself on half hour |
| | before first patient is |
| | expected |
| 2. | Send notice to front desk if not |
| | receiving constant power or not |
| | fully charged |
| 3. | Automatically download medical |
| | records from EHR system for |
| | patients expected that day. |
| | Medical records will include |
| | pictures, SOAP notes, complete |
| | chart, facial recognition data |
| 4. | Encrypt downloaded information |
| | as appropriate for HIPPA |
| | compliance |
| 5. | Kiosk checks regularly (e.g., |
| | hourly) whether any new |
| | patients have been booked and |
| | download the medical records |
| | for those new patients |
| 6. | Send confirmation to front desk |
| | of tasks completed and confirm |
| | Kiosk ready to interact with |
| | patients scheduled to be |
| | examined today |
| 7. | Enable local operation of EHR |
| | system - in case of internet or |
| | power interruption. Provide |
| | confirmation that backup EHR |
| | system function is operational |
| 8. | If cleaning crew was not |
| | detected overnight or any other |
| | suspicious activity that was |
| | detected overnight - generate |
| | appropriate alerts |

TABLE 2
Illustrative Patient Check-in Workflow

| Step No. | Task Description |
|---|---|
| 1. | Greet patients - scheduled |
| | appointments or walk-ins, |
| | patients who show up early to an |
| | appointment, guardian or other |
| | special cases |
| 2. | Identify/authenticate patient - |
| | biometrics or in tandem with |
| | patient's smart phone |
| 3. | Capture demographics/change in |
| | demographics from patient |
| 4. | Take picture(s) of the patient |
| | or extract picture from |
| | driver's license |
| 5. | Capture medical history and ask |
| | if any changes in medical |
| | history if > 6 months from last |
| | visit |
| 6. | Confirm/capture pharmacy |
| | information |
| 7. | Scan insurance card and |
| | driver's license |
| 8. | Scan any other documents |
| | patient might have brought |
| | (e.g., pathology reports from |
| | other practices) |
| 9. | Promote and enroll patient in |
| | appointment reminder services |
| | or other beneficial programs |
| 10. | Obtain consents (HIPAA, |
| | financial, guardianship) |
| 11. | Obtain copy of referral if |
| | required from patient, confirm |
| | validity (date, name, |
| | procedure, CPT code), check for |
| | insurance authorization |
| 12. | Collect past due balance, |
| | copay, coinsurance, deductible |
| | or other fees (credit card, |
| | check, cash, Venmo, Zelle), |
| | record payment in EHR system |
| 13. | Mention office specials on |
| | products, procedures |
| 14. | Advise patient on approximate |
| | waiting time |
| 15. | Advise patient on reasons for |
| | expected delay - just enough |
| | information that complies with |
| | HIPPA rules, but enough to |
| | generically explain reason for |
| | delay to waiting patients |
| 16. | Ask if patient has any questions |
| | or needs any help |
| 17. | Instruct patient to have a seat |
| | until called to mid-office |

TABLE 3
Illustrative Medical Assistant Workflow

| Step No. | Task Description |
|---|---|
| 1. | Room patient - Show map on kiosk |
| | screen or show directions to |
| | exam room. Direct patient using |
| | another kiosk or showing |
| | information on patient's mobile |
| | device. |
| 2. | Kiosk in exam room will |
| | recognize/greet patient as |
| | enters exam room |
| 3. | Kisok will communicate that |
| | patient can mute to interact via |
| | touch or text or unmute at any |
| | time to change to voice |
| | interaction or toggle back and |
| | forth |
| 4. | Kiosk will ask for patient's |
| | chief complaint (if not already |
| | asked) and if already asked, |
| | prompt for any additional |
| | complaints |
| 5. | If Kiosk does not recognize a |
| | patient prompt for biometric |
| | authentication, confirm the |
| | identity of patient or guardian |
| 6. | Kiosk provides standard |
| | instructions (take out gum, put |
| | away phone, sit on chair), take |
| | off clothes and where to put |
| | clothes for full body exam and |
| | put on medical gown |
| | Disable camera until it receives |
| | a voice prompt from a patient |
| | that they are ready for |
| | examination |
| 7. | Kiosk confirms patient pharmacy |
| | information |
| 8. | Kiosk confirms patient medical |
| | history |
| | Use QR code to activate any |
| | device as display screen or |
| | input device |
| | Prompt patient for answers to |
| | MIPS questions (give patient |
| | choice of voice or touch |
| | interaction) |
| | Adjust MIPS questions as per |
| | diagnosis or chief complaint(s) |
| 9. | Capture vital signs |
| 10. | Check if any reports (labs, |
| | paths) need to be reviewed |
| 11. | Confirm current medications, |
| | update as needed |
| 12. | Obtain chief complaint (perhaps |
| | multiple) |
| | Ask common questions and record |
| | answers. |
| | Provide access to FAQs. |
| 13. | Talk about in-house |
| | products/specials and take |
| | orders |
| 14. | Monitor patient until provider |
| | enters room |
| | Summon emergency help if need be |
| | Call 911 on command |
| | Alert office staff |
| 15. | Make certain has referral if |
| | needed |
| | Pre-auth for future |
| | procedure(s)/visit(s) |
| 16. | Take pictures (use Kiosk camera |
| | or camera on mobile device or |
| | provider/patient) as needed of |
| | ailment or biopsy |
| 17. | Obtain written and/or verbal |
| | consents as needed - to be part |
| | of patient medical record |
| 18. | Ask the patient if ready for the |
| | provider and send a message to |
| | medical assistant/provider that |
| | the patient is ready to be seen |
| 19. | Alert staff that the patient is |
| | ready to see the provider. |
| | Instruct the patient that the |
| | medical assistant/provider will |
| | knock before entering the exam |
| | room |
| 20. | Prepare/calm patient verbally |
| | for Biopsy or other procedure as |
| | needed |
| 21. | Provide access to educational |
| | materials regarding scripts |
| | prescribed/procedures to be |
| | performed during visit |
| 22. | Access third-party vendors to |
| | estimate prescription drug costs |
| 23. | Arrange for cab/elder transport |
| | if required |
| 24. | Comfort patient, play desired |
| | music or other personal requests |
| 25. | Remind patients to follow-up if |
| | needed |
| | Provide information on follow up |
| | care |
| 26. | Alert office staff to clean room |
| | and prepare for next patient |
| 27. | Direct patient to check out - |
| | escort patient to front desk |
| | Display or print map with |
| | directions to check out |

TABLE 4
Illustrative Checkout Workflow

| Step No. | Task Description |
|---|---|
| 1. | Recognize/greet patient |
| 2. | Review charges/balance |
| 3. | Collect payment |
| 4. | Enroll patient in automated |
| | payments |
| 5. | Set up any payment plan |
| 6. | Dispense any purchased |
| | product(s) |
| 7. | Provide summary (text, video) of |
| | care (on paper) or email/text |
| 8. | Provide printed |
| | prescriptions/instructions if |
| | requested/needed or email/text |
| 9. | Provide any requested |
| | educational material - |
| | Printed/email |
| 10. | Complete satisfaction survey of |
| | current visit |
| | Online platforms |
| | Capture audio/text/video |
| | testimonials or feedback |
| 11. | Schedule follow up |
| | appointment(s) |
| | Inform patient if new referral |
| | will be needed |
| | Email/text reminders regarding |
| | appointment and referral |
| 12. | General statement of the day |
| | regarding weather, pollen count, |
| | traffic |

TABLE 5
Illustrative Scheduling Workflow

| Step No. | Task Description |
|---|---|
| 1. | Schedule/reschedule/cancel/confirm |
| | appointments based on patient |
| | needs, provider needs or office |
| | needs |
| | Encourage patients to accept |
| | specific slots |
| | Determination which slots to |
| | prioritize may be based on AI model |
| 2. | Contact patient if new slot becomes |
| | available |
| 3. | Integrate with office's online |
| | scheduling system |
| 4. | Check authorizations and pre- |
| | authorizations |
| 5. | Create new patient records with |
| | captured demographics (e.g., |
| | email, mobile number, address) |
| 6. | Send intake/confirm links to |
| | patients |
| 7. | Add to waitlist and dynamically |
| | contact patient as desired |
| | appointment slots open up |

TABLE 6
Illustrative Analytical Utilities

| Step No. | Task Description |
|---|---|
| 1. | Pull statistics regarding time |
| | spent with each patient |
| 2. | When scheduling appointments, |
| | adjust time allotted based on |
| | patient's previous visits or other |
| | patients with similar conditions or |
| | current office conditions (e.g., |
| | number of patients waiting to be |
| | seen, number of providers |
| | available, time of day) |
| 3. | Upsell - review a patient's medical |
| | history and promote target |
| | procedures, products and services |
| | based on various medical, personal |
| | and demographic criteria |
| | Illustrative criteria may include |
| | Fitzpatrick skin type, size of |
| | pores, shape of face, and type of |
| | skin (e.g., dry, oily, scaly) |
| 4. | Measure and record tasks and time |
| | spent by each provider |
| | Such statistics may be used to |
| | detect fraud by correlating |
| | procedures to time spent with |
| | patients or number of patients seen |
| | per day |

TABLE 7
Illustrative Employee Workflow

| Step No. | Task Description |
|---|---|
| 1. | Identify person as employee or |
| | office vendor |
| 2. | Locate records associated with the |
| | identified person (these records |
| | should preferably be stored locally |
| | on kiosk) |
| | These records include information |
| | about an employee/vendor work |
| | schedule |
| 3. | Register time employee or vendor |
| | (e.g., cleaning service) checked in |
| | and checked out |
| 4. | Prompt for whether there were any |
| | problems, or any supplies are |
| | needed |
| | Kiosk may be configured to self- |
| | order supplies (e.g., from online |
| | marketplace) or notify registered |
| | point person - may be different for |
| | each detected employee or vendor) |
| 5. | If an employee arrives/leaves early |
| | or late - check whether lateness or |
| | early has been authorized or prompt |
| | for other requested office |
| | responses (for example only ask if |
| | the employee has been late more |
| | than 3 times) |
| | Record employee responses to |
| | prompts |
| 6. | Generate reports as requested by |
| | office |
| 7. | Connect to payroll provider and |
| | upload captured data |

TABLE 8
Illustrative Office Close Workflow

| Step No. | Task Description |
|---|---|
| 1. | Ideally, store 6-weeks of patient |
| | photos locally on kiosk |
| | Kiosk may periodically purge stored |
| | photos to manage disk space |
| 2. | Make sure all patient data entered |
| | during the day has been saved |
| | locally and pushed to cloud based |
| | EHR system |
| 3. | Place kiosk in surveillance mode |
| | overnight to monitor any people |
| | coming and going after hours |
| 4. | Delete locally stored patient |
| | records and biometric data as |
| | needed (every three days, 3 weeks |
| | or 3 months) |
| | Check to make sure that data to be |
| | deleted has been saved in EHR |
| | system |
| 5. | Make sure kiosks are plugged in |
| | overnight |

Deployment of one or more kiosks may provide a medical office with functional advantages. Kiosks may improve compliance with regulatory requirements and maximize reimbursement rates. Kiosks may provide patients with better access to better information. Kiosks may increase patient interaction with service, specials, products available at an office or healthcare facility.

The kiosk may allow offline operation. During a power/internet outage, all other computers may be offline. The kiosk may store critical medical data locally and allow an office to continue seeing patients, data being stored, and payments registered. Even during a power/internet outage, the battery powered kiosk may remind a provider to follow up on a critical diagnosis After power comes back on, data stored locally on the kiosk may be pushed to the cloud and integrated into an EHR system.

Kiosks may improve clinical care. They may present patients education video before/after procedures, provide education on medication and diagnosis after a visit auto send information to a patient's email, send medication reminders if desired, allow patients to submit questions beforehand, calm patients by explaining procedure and provider credentials, and what to expect during examination.

Kiosks may improve office efficiency by automating the movement of patients into the exam room and obtaining medical history, explaining post visit care, allowing providers to leave and see another patient, knowing which patient is ready to be seen. Kiosks may help reduce risk of non-compliance by making sure patients schedule follow up appointments, fill out forms completely (medical history, social history, personal history).

Kiosks may enhance privacy and personalization. Kiosks may greet patients by name, recognize and authenticate patients before divulging confidential information.

Apparatus may include automated an voice-based appointment booking system. The appointment booking system may include one or more features of a computer system. The appointment booking system may provide super-efficient and natural interaction for booking appointments. Appointment scheduling is at the intersection of efficiency and timely access to health services. Timely access to healthcare services is important for realizing good medical outcomes.

Only three out of 10 consumers who try to book a healthcare appointment online will succeed. It has been reported that more than half of patients are going without care access because the online appointment scheduling systems they rely on are not fulfilling their needs and expectations. Particularly, many patients report that they did not access healthcare in the past year because using an online self-scheduling system was too complicated. In one survey, 70 percent of patients reported that they began booking an appointment using an online self-scheduling tool or patient portal and were eventually referred to a telephone call with a call center staffer to finish booking. Traditionally, booking an appointment over the telephone has been a long-winded and inefficient process.

On the other hand, humans naturally communicate using voice and it is an intuitive and fast method of communication. Apparatus described herein provide an automated voice booking system that is faster and more intuitive than online booking platforms. The voice-based system described herein eliminates waiting to connect with someone on the phone, getting placed on hold, or experiencing mistakes about available appointments or which insurance is covered.

The voice-based appointment booking system described herein is HIPPA compliant. The voice-based appointment booking system operates (e.g., respond to caller input, access and present information extracted from an EHR system) within time and performance limits that allow appointments to be booked in under five minutes and preferably under 3 minutes. The voice-based appointment booking system may mimic live human-to-human interaction and provide an institute comfortable interface for callers.

The appointment booking system may include a middleware system (Layer 2) that interacts rapidly with an IVR system (Layer 1) that answers phone calls and integrates with one or more EHR systems (Layer 3). The System collectively includes the IVR system (Layer 1), Middleware (Layer 2) and integration to the various EHR systems (Layer 3).

The appointment booking system may provide intuitive and natural language voice functionality that substitutes for caller interaction with a live, human agent. The system may provide callers with voice prompts using natural, human sounding voices and in multiple languages. After initiating a voice-based conversation, the IVR system may facilitate bi-directional interaction with the caller. The IVR system may allow the caller to shift back and forth between a text/video-based conversation and voice-based conversation.

The AI model may understand callers when they speak in full sentences using natural language. The AI model may decipher caller voice inputs. The AI model may machine-generate responses in lifelike speech to the caller voice inputs. The AI model may provide machine-generated responses that sound humanly conversational and include typical human fillers (such as “uhh” or “hmmm”), expression and intonation.

The middleware system may apply filtering rules. The filtering rules may be customized by each office. The filtering rules may impact various stages of the appointment booking process. The filtering rules may apply business intelligence logic for reducing no shows and optimizing scheduling. The filtering rules may encourage patients to book appointments that are optimal for both patient and office. The filtering rules may attempt to fill appointment slots on target days/times and provide callers with desired appointment slots.

The filtering rules may take account of providers available at an office location, time of day, and local factors such as weather and current traffic conditions. The filtering rules may vary overbooking rates based real-time or historical data. An illustrative filtering rule may generate medical appointment choices for a caller based on: available appointment slots, office location(s), whether a caller is a new or established patient, medical insurance coverage, desired provider (doctor, MA, PA surgeon), desired date and time of appointment, and patient/caller demographic information. Other customer filtering rules may be configured by an office.

The appointment booking system will be (Layers 1, 2 and 3) will be HIPPA compliant. The appointment booking system may integrate with one or more EHR systems. Each EHR system integration may allow the middleware system to access patient and scheduling information using industry standard technologies that provide real-time bi-directional communication with the EHR system (Layer 3) and patient data stored therein.

The integration with an EHR system may allow the middleware system to extracts patient information, push updates to patient information, extract available appointment slots over a defined time interval. All components of the appointment booking system (Layers 1, 2 and 3) operate (e.g., respond to caller input, access and present information extracted from an EHR system) within time and performance limits that are commercially reasonable for a system whose goal is to mimic live human-to-human interaction.

The appointment booking system may provide an administrative interface. The administrative interface provides user access for configuring custom filtering rules that define operating and scheduling optimization criteria. Illustrative filtering rules may control available appointment slots presented to a caller based on: providers that work at the dialed office location, patient age or other restrictions on which type of patients can be examined by those providers, insurance accepted by those providers, ramp, elevator or other accessibility features available at the dialed office.

Monitoring caller behavior and performance metrics is valuable when evaluating the effectiveness of an appointment booking system. Monitoring performance metrics enables healthcare providers and institutions to identify areas for improvement, optimize their scheduling systems, and enhance patient satisfaction.

The appointment booking system may log/track caller behavior and performance metrics for each layer. For example, with respect to the IVR system (Layer 1), the system may track and log: duration of each call, transcript of each call, how many times has the caller dialed into the system, resolution of the phone call, abandonment rate (when and how often do callers leave the IVR system before completing a task), transmission latency when communicating with the EHR systems.

The appointment booking system may include a dashboard for visualizing tracked statistics and reports based on analysis of logged/tracked caller behavior and system performance metrics. In parallel with voice call, the system may send text messages or pictures of information being discussed on call. For example, the IVR system may send a caller a message (text or picture) showing various appointment choices that are available for selection. Or, in tandem with voice call, the IVR system may initiate a video conference with a caller.

The system may be configured to send automated voice reminders about upcoming appointments. The system may allow patients to interact with the IVR system to confirm, reschedule or cancel an appointment. During a reminder call, the patient may be provided access to office information or any other IVR system options and menus.

The AI model may dynamically adjust voice properties used to interact with a caller based on detected caller voice input. The AI model may dynamically determine which voice or language should be used to interact with the caller. This may dynamically change during the conversation. Illustrative voice properties that may be dynamically adjusted by the AI model may include male, female, accent, tenor, cadence, language, volume, spunk, humor, vocabulary, mirror caller response or if detect that caller is getting upset, slow down the voice output responses or select voice properties configured to deescalate the tension.

During a video call, the AI model may adjust voice output properties based on how the caller looks, caller motions or how caller speaks on video. Based on content of caller voice input, the AI model may adjust voice output properties. For example, if a caller says something unfortunate occurred, the AI model may generate voice output that employs a sympathetic voice or presents sympathetic message.

The middleware system may communicate with the EHR system using APIs. If API access to an EHR system is not available, the appointment booking system may still provide caller with available appointment slots by extracting data from screenshots of schedule displayed in EHR system to identify open appointments slots. This will allow patients to schedule appointments using voice regardless of functionality provided by the EHR system.

The appointment booking system may accurately confirm caller information such as name, phone number, birthdate and insurance. Accurate capture and confirmation of such information may reduce a number of duplicate patient records created when booking appointments over the phone or even when booking online. Unintentional duplicate charts happen when a patient or office staff member enter names incorrectly. Duplicate charts are hard to both find and merge so the provider may see an incomplete record.

Using the appointment booking system, a caller may be presented with a proposed spelling of their name and asked to confirm the spelling. The confirmation may be presented to the caller via voice, text message or video chat. The AI model may apply fuzzy logic to identify and eliminate potential duplicate patient records. The appointment booking system may generate office forms and pre-populate those forms with confirmed spellings of patient name, insurance and other information that has been provided by a caller over the phone.

The appointment booking system may verbally explain to a caller why a presented appointment slot is advantageous. For example, the system may explain that there is expected to be nice weather on the day or time of the available appointment. Or, the system may explain that traffic is expected to be light based on the presented appointment day/time and expected travel path the caller will take from their home or work address to the dialed office. Or, the system may explain that a wait time is expected to be short for the presented appointment.

A caller may be prompted to provide a voice input explaining why they are scheduling, rescheduling or cancelling an appointment. The captured voice input may be processed to determine how to classify the appointment or determine a duration of the appointment. For example, based on the provided reason, the AI model may determine whether a caller has scheduled a medical or cosmetic appointment. The AI model may identify a provider that has the expertise to service the medical needs of the caller.

The AI model may generate various personas and create competition among those personas to achieve the highest number of appointment bookings within a defined timeframe. The persona interacting with a caller may ask the caller to complete a survey and explain that completing the survey will help the persona win the competition against the other personas.

The IVR system may allow a provider or other office staff member to record voice input describing their schedule. The AI model may generate a schedule based on the recorded voice input describing the schedule. The generated schedule may then be synced and integrated with the EHR system. For example, a provider may speak to the IVR system “I will see patients on Monday, Wednesday and Friday from 10 am-1 pm. The IVR system may pass the captured voice input to the AI model. The AI model may generate the described schedule so that patients are only booked for the given provider during the described days and times.

If a provider needs to cancel appointments, a message can be sent to the IVR system. The IVR system may automatically trigger phone calls to those patients who need to be rescheduled or informed of the delay/cancellation.

Based on which provider is associated with the appointment selected by a caller, the caller may be played a personal audio or video message from the provider they are expected to visit. The provider message may give the caller a personal greeting at conclusion of an appointment booking call.

The IVR system may prompt a caller to complete a satisfaction survey after concluding a caller interaction. Prompting callers to complete a satisfaction survey after concluding a caller interaction is an important step in evaluating patient satisfaction and identifying areas for improvement. This feature can help healthcare providers refine their scheduling systems and enhance the overall patient experience.

A major technical challenge to AI model reliability is its tendency to “hallucinate” and produce unreliable responses to inputs. The appointment booking system solves this technical problem with a hybrid solution that leverages use of an AI model with a structured IVR system that does not rely on machine learning algorithms for decision making. The AI model may check the IVR system and vice versa to ensure reliable and natural sounding healthcare applications.

The overall goal of the IVR system is to provide natural, human sounding voices (e.g., voice speed, female, male, with local accents) and interact with callers using multiple languages, over multiple channels (voice, text, video) to book/change appointments, request office information and complete other self-service tasks. Interaction with caller will begin by receiving voice or text input to the IVR system and prompting the caller to choose a self-service task (e.g., appointment booking). Verification of the appointment details may be sent to the patient after booking the appointment

The IVR system may utilize input provided by the caller to provide personalized responses (e.g., use caller's first name or show understanding of the task the caller wants to complete) and overall mimic a personal human-to-human interaction. The IVR system may prompt caller to provide necessary voice input to execute a desired appointment task. For example, if the caller wants to book an appointment, IVR system may prompt for caller's name, address, birthdate, desired provider, desired office location, insurance information.

The IVR system may pass information captured from each caller to the middleware system (Layer 2). The middleware system may use the information captured by the IVR system (Layer 1) to interact with the EHR system (Layer 3) and apply filtering rules that are specific to the medical practice or office the caller has dialed. The IVR system may log data elements for later analysis.

Illustrative data elements that need to be tracked and collected include: time each call is received, calling and dialed number, whether call received was from a landline or mobile number, number of abandoned calls and at what point in the workflow was the call abandoned (e.g., in response to which question or prompt), duration of each call, time delay between IVR voice prompts and receiving caller input, voice type/language/gender used for each call (e.g., male voice speaking Spanish), number of appointments booked, changed or cancelled (per hour, day, week, provider, office location, etc.)

In addition to using natural language, the IVR system may utilize multiple communication channels to interact with the caller. The alternative communication channels will be dynamically selected based on the context of the conversation and how a caller would most efficiently process information. For example, appointment choices may be more efficiently presented to callers visually rather than audibly. Therefore, while a phone call is in progress, the IVR system may text the caller appointment choices and then have the caller respond (audibly or touch) with the choice they want to select. IVR systems may also ask whether the caller would prefer to hear choices audibly or visually—for example the caller may be driving or have a visual disability and prefer to hear the choices audibly.

After booking an appointment, text the caller a summary about the appointment. After a caller books an appointment, the IVR system may text the caller links to office forms that need to be filled out before the scheduled appointment. The text message may include a link for the caller to upload an image of their insurance card or driver's license. Information extracted information from the image of the insurance card or driver's license would then be used by the middleware system to formulate EHR system (Layer 3) requests.

The middleware system (Layer 2) may define operating and optimization criteria for the appointment booking system based on specific business rules. This feature allows for tailored solutions that cater to the unique needs of each medical practice or organization. The appointment booking system aims to facilitate rapid appointment booking in under 5 minutes (preferably under 3 minutes) for patient record matching, insurance clearance and treatment-targeted booking. To achieve this the system incorporates custom filtering rules native to the specific requirements of each medical practice or organization.

The filtering rules may impact various stages of the appointment booking process. The middleware system may be coupled with an AI model to In addition to the fuzzy logic component to reduce duplicate patient records referenced earlier, factors include: business intelligence and rules for reducing no shows and optimizing scheduling, matching office schedule optimization with factors desirable by the caller, providers available at an office location, time of day, and local factors such as weather and current traffic conditions and overbooking allowances based real-time or historical data.

The middleware system may be coupled to an AI model. The AI model may dynamically respond to fluctuations in the real-time availability of open appointment slots for an office location at a given time of day. The AI model may also account of factors local to a caller, such as weather and traffic conditions and the caller's ability to reach the office during their desired appointment time.

The middleware system may assess the caller's insurance coverage to confirm provider coverage and suggest providers who accept the same plan to reduce potential financial burdens. The middleware system may apply filtering rules to provide callers with appointments choices with providers that are available at a dialed office location, at the caller's desired appointment date/time and accept the caller's insurance. The middleware system may also apply filtering rules that account for provider age or other restrictions when attempting to match a caller with available appointment slots.

Other filtering rules defined by office-specific requirements may include: provider specialties, ramp or elevator accessibility, and insurance accepted by providers at the dialed office location to optimize scheduling and reduce no-shows. A flexibility of the middleware system to connect the IVR system and leverage decision making of an AI model and filtering rules set based on office-specific requirements maximizes the caller experience and practice efficiency.

The middleware system may determine which office location to associate with each caller. The middleware system may determine an office location based on a phone number dialed by caller. The middleware system may instruct the IVR system to prompt the caller to choose a specific location (e.g., if caller dialed general office number, corporate office or 800 number). An AI model associated with the middleware system may assign an office location to the caller based on office locations previously visited by caller.

If a desired appointment is not available, the middleware system may apply a filtering rule to offer alternatives based on patient wishes and office needs. The IVR system may provide voice output to the caller explaining that the desired appointment is not available and offer 1-3 alternative appointment options. The middleware system may select the alternative appointment options. If very few appointments are available at a target office location, a filtering rule may switch to a different office location within a predefined radius of the dialed office location.

The middleware system may classify caller as a “new” patient (no patient record in EHR) or as an “established” patient (successfully locate patient record in EHR). A filtering rule applied by the middleware system may determine how often to ask callers to update insurance information. The middleware system may apply filtering rules based on an appointment type. Illustrative appointment types may include: medical, non-medical (suture removal, follow up wound care), surgery, cosmetic and telehealth. The IVR system may prompt a caller to provide an appointment type. In some embodiments, the AI model may determine appointment type based on a natural language reason for an scheduling an appointment provided by the caller.

The middleware system may define a time range that will be searched for open appointments in an EHR system (Layer 3). The exact appointment requested by a caller may not be available. The middleware system may automatically determine relevant alternatives based on input already captured from the caller. For example, if there are no afternoon appointments with Dr. Z on Thursday, the middleware system may present an alternative-such as an appointment with Dr. X on Thursday afternoon or with Dr. Z on Wednesday morning.

The middleware system may determine which appointments are presented to a caller based on a caller's insurance plan information. The middleware system may only search the EHR system for appointments with providers that participate in the caller's insurance plan. In some embodiments, the caller may not be prompted to specify a desired time for an appointment. The middleware system may automatically search for available appointments based on attributes of a caller's last three appointments. Based on the last three appointments, the middleware system may determine a template for booking a new appointment for the caller.

The middleware system may determine whether to present target information to caller about consequences of cancelling an appointment (e.g., whether cancelling the appointment will incur a fee) and instruct IVR system (Layer 1) to inform caller of those consequences. The middleware system may determine whether a caller should be blocked or restricted from booking, rescheduling, or cancelling an appointment. In determining whether to impose a restriction, the middleware system may take account of: number of reschedules or cancellations by the caller in the last month, the reason for the current appointment, current outstanding balance (outstanding balance will need to be pulled from EHR system).

Via the IVR system, the middleware system may receive an appointment selection from the caller and trigger syncing of any newly scheduled, changed, or cancelled appointments with the EHR system. In response to a successful booking, the middleware system may provide the IVR system with access to forms that need to be completed by the caller before their appointment or other pre-appointment instructions. The middleware system may trigger the IVR system to send appointment reminders at a predetermined time before an upcoming appointment.

The middleware system may determine whether an office billing department or call center is open before transferring a caller to that department. The middleware system may coordinate callbacks for the specific department.

The middleware system may coordinate bi-directional communication with the EHR systems (Layer 3). Such bi-directional communication may include: pulling open appointments slots, confirming new appointments, pushing newly booked, rescheduled or cancelled appointments, pulling patient medical records, updating patient medical implement records, appointment waitlist functionality by periodically checking an EHR schedule, locating appropriate open appointment slots for a caller, ability to accept payments over the phone or via text, automated verification of insurance information, providing an administrator interface to customize filtering rules.

The middleware system may provide an intuitive, easy to use administrative user interface for office personnel to set values for custom filtering rules. Illustrative filtering rules that may be set for each office location include: default appointment scheduling rules (e.g., break times, when to schedule new patients), weekend, holiday, walk-in scheduling rules, appointment categories and service provided, whether or not a fee is applied for cancellation within 24-hours and amount of the fee, specific diagnosis(es) or procedures treated at target location, provider attributes (specialties, participating insurance, available locations, hours and schedule, patient type restrictions).

The appointment booking system may integrate with an EHR system. The integration with an EHR system provides real-time bi-directional communication that: extracts patient information stored in the EHR system, pushes updates to patient and schedule information, extracts open appointments slots over a defined time interval from the EHR system, and updates the schedule maintained by the EHR system.

The integrations to EHR systems provide the ability in real-time for the middleware system to pull open appointments from each EHR system, search for a target appointment requested by a caller, locate a patient record and book an appointment in response to a caller selection, push newly booked, rescheduled or cancelled appointments to each EHR system, based on information obtained from a caller (e.g., name, phone number, birthdate) search for and obtain a corresponding patient record.

When the EHR system contains a patient record for a caller, the middleware system may obtain the following information from the EHR system: patient ID (as used by EHR system), patient Name (First, Last), birthdate, gender, upcoming open appointment(s) (if any), insurance information, attributes of caller's three most recent appointments (provider, location, day of week, time and duration), payment information (payment on file and current balance), most recent diagnosis special patient needs (disability or language requirements), other information as may be available from the EHR system.

Illustrative embodiments of apparatus and methods in accordance with the principles of the invention will now be described with reference to the accompanying drawings, which form a part hereof. It is to be understood that other embodiments may be utilized, and structural, functional and procedural modifications may be made without departing from the scope and spirit of the present invention.

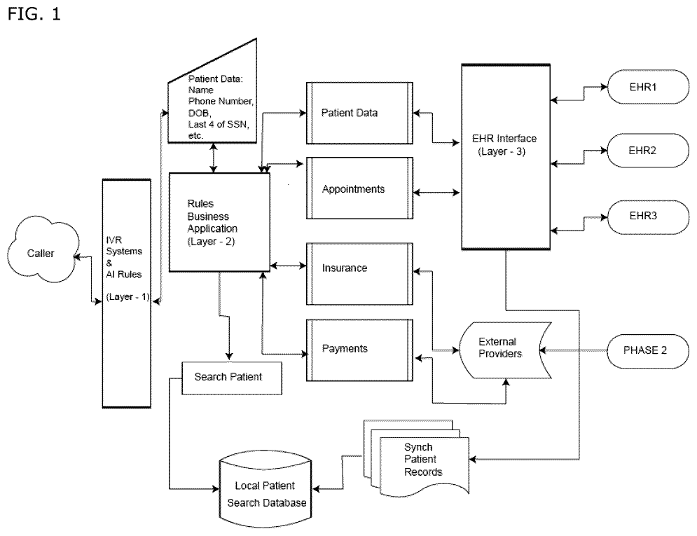

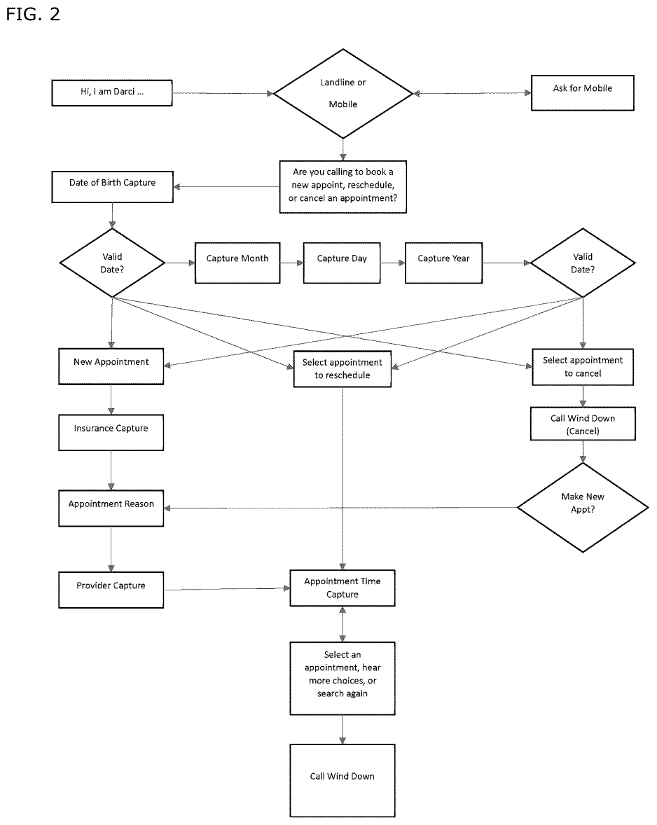

FIG. 1 shows an illustrative system architecture in accordance with the principles of the description. FIG. 2 shows an illustrative process that may be implemented by an appointment booking system in accordance with the principles of the description.

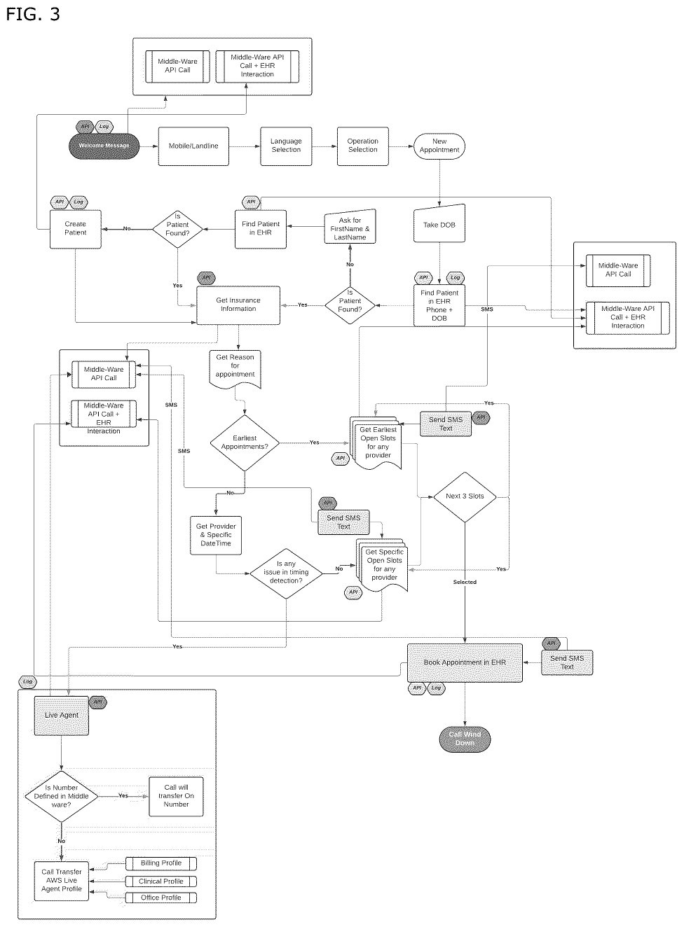

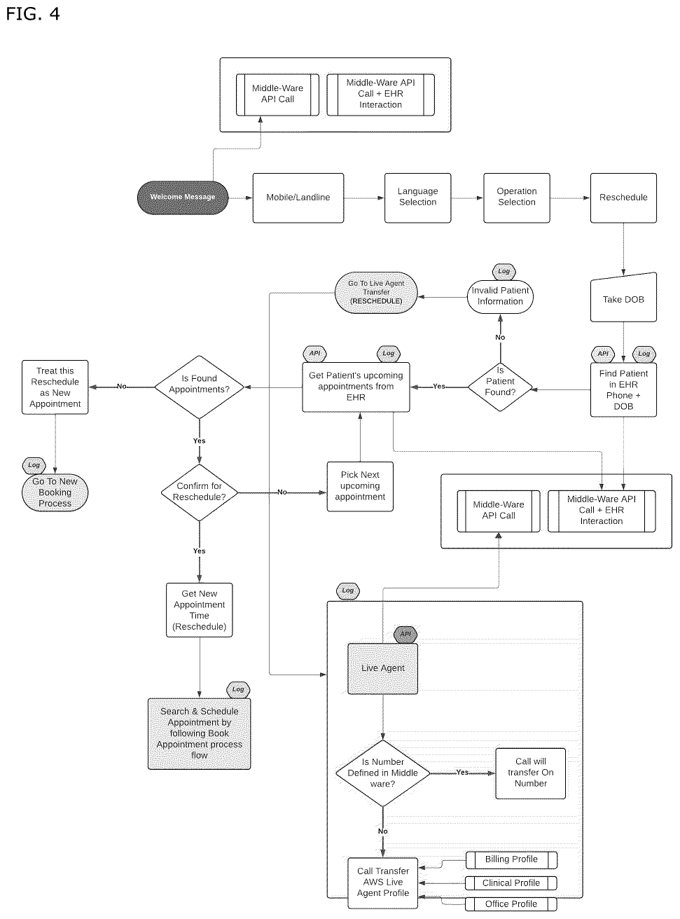

FIG. 3 shows an illustrative process for booking a new appointment, components and functions that that may be implemented by an appointment booking system in accordance with the principles of the description. FIG. 4 shows an illustrative process for rescheduling an appointment, components and functions that that may be implemented by an appointment booking system in accordance with the principles of the description. FIG. 5 shows an illustrative process for cancelling an appointment, components and functions that that may be implemented by an appointment booking system in accordance with the principles of the description.

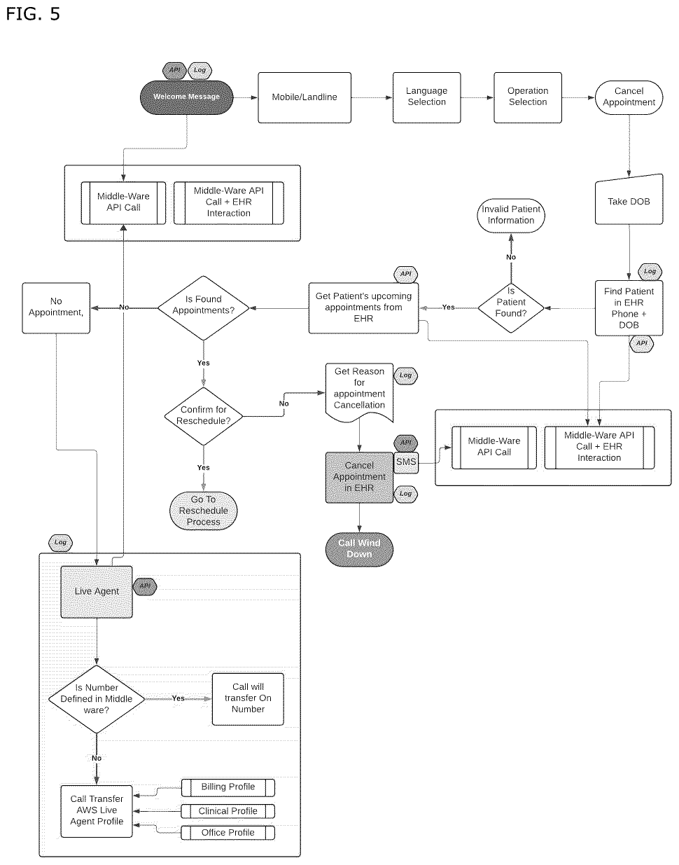

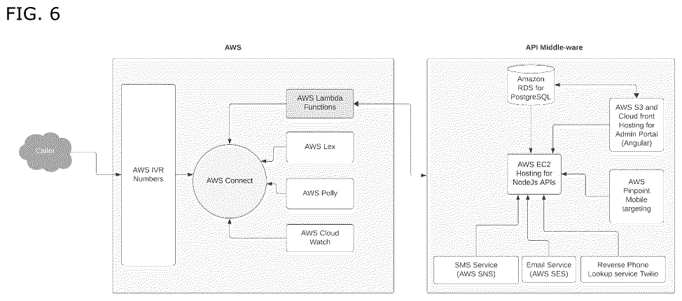

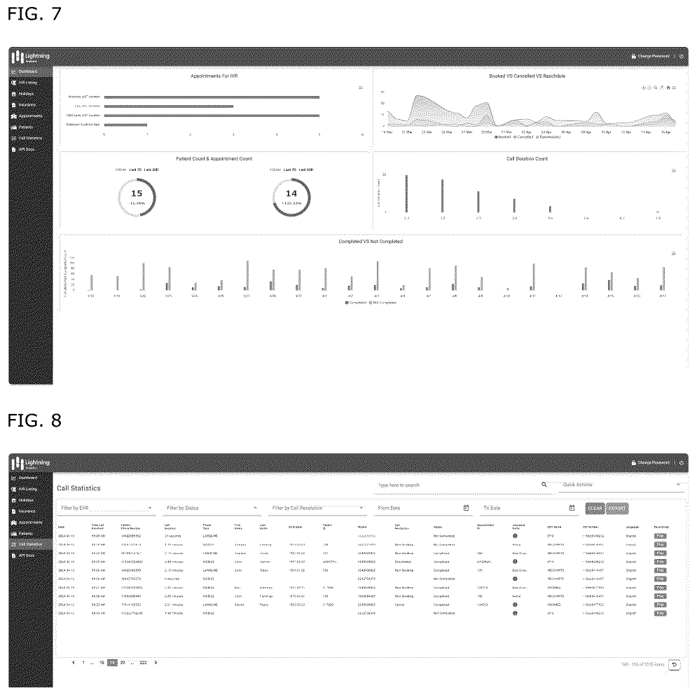

FIG. 6 shows an illustrative system architecture for an appointment booking system in accordance with the principles of the description. FIG. 7 shows an illustrative graphical user interface for viewing activity tracked by an appointment booking system in accordance with the principles of the description. FIG. 8 shows another illustrative graphical user interface for viewing activity tracked by an appointment booking system in accordance with the principles of the description.

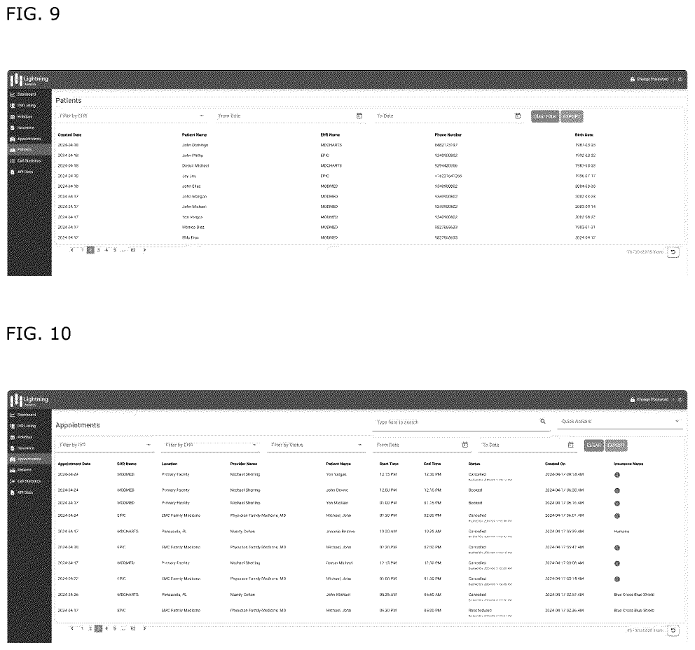

FIG. 9 shows an illustrative graphical user interface for viewing new patients that have been registered by an appointment booking system in accordance with the principles of the description. FIG. 10 shows an illustrative graphical user interface for viewing appointment activity processed by an appointment booking system in accordance with the principles of the description.

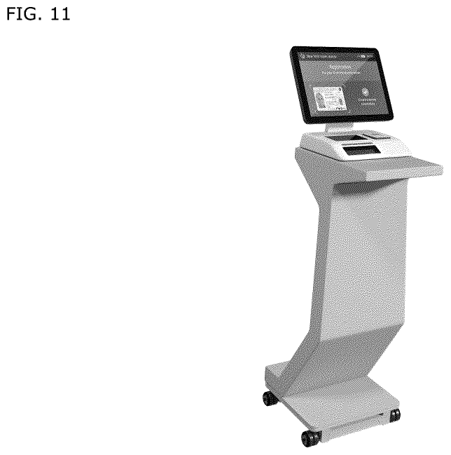

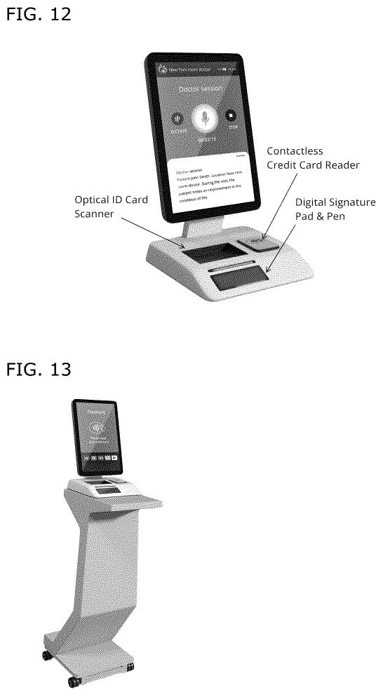

FIG. 11 shows an illustrative kiosk affixed to a stand in accordance with the principles of the description. FIG. 12 shows an illustrative kiosk and associated peripheral devices in accordance with the principles of the description. FIG. 13 shows an illustrative kiosk positioned on a stand in accordance with the principles of the description.

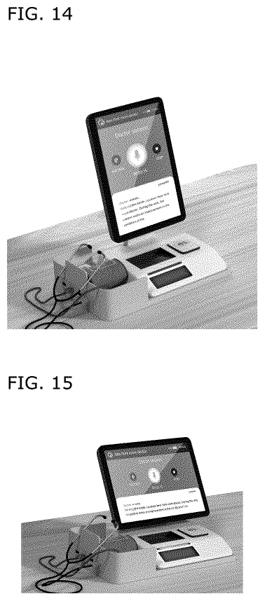

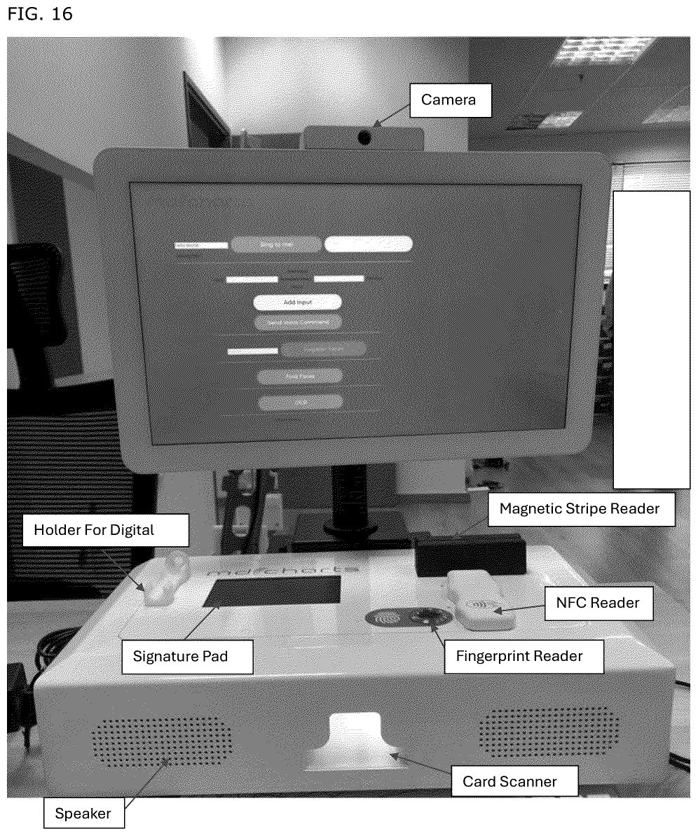

FIG. 14 shows an illustrative kiosk, a docking station and associated peripheral devices in accordance with the principles of the description. In FIG. 14, a screen of the kiosk is in a portrait orientation. FIG. 15 shows an illustrative kiosk, a docking station and associated peripheral devices in accordance with the principles of the description. In FIG. 15, a screen of the kiosk is in a landscape orientation. FIG. 16 shows an illustrative kiosk and components in accordance with the principles of the description.

Thus, apparatus and methods for ARTIFICIAL INTELLIGENCE PATIENT INTAKE AND APPOINTMENT BOOKING SYSTEM are provided. Persons skilled in the art will appreciate that the present disclosure can be practiced in embodiments other than the described embodiments, which are presented for purposes of illustration rather than of limitation.

What is claimed is:

1. A system for reducing hallucinations in software that incorporates artificial intelligence (“AI”), the system comprising:
an interactive voice response (“IVR”) system configured to:
receive a voice input from a caller; and
compute a first intent of the caller from the voice input;
an artificial intelligence (“AI”) model that is configured to receive the voice input and compute a second intent of the caller from the voice input; and
a middleware system that is configured to formulate a response to the caller based on the first intent and the second intent.

2. The system of claim 1 wherein based on a confidence measure associated with the first intent, the middleware system is configured to throttle a level of regularization applied to the AI model.

3. The system of claim 1, wherein the middleware system is configured to pass the response to the IVR system and the IVR system is configured to generate audio output based on the response.

4. The system of claim 2 wherein the middleware system is configured to increase the level of regularization based on a threshold confidence measure associated with the first intent.

5. The system of claim 2 wherein the middleware system decreases the level of regularization when the first intent is above a threshold confidence measure.

6. The system of claim 1 wherein the middleware system is configured to:
instruct the AI model to compute the second intent before the IVR system computes the first intent;
compute a confidence measure associated with the second intent; and
when the confidence measure associated with the second intent is below a threshold level, instruct the IVR system to compute the first intent.

7. The system of claim 1 wherein the middleware system is configured to:
instruct the IVR system to compute the first intent;
assess a confidence measure associated with the first intent; and
when the confidence measure associated with the first intent is below a threshold level, instruct the AI model to compute the second intent.

8. The system of claim 1 wherein the voice input is a natural language statement of the caller.

9. The system of claim 1 wherein the AI model is configured to recursively train itself using a plurality of first intents generated by the IVR system.

10. The system of claim 1 wherein:
the AI model is configured to generate a plurality of intents in response to a prompt; and
the IVR system is configured to determine whether the voice input matches one of the plurality of intents.

11. The system of claim 1 wherein, the AI model is configured to activate the IVR system and the middleware system in response to detecting a pre-defined trigger event.

12. The system of claim 11, wherein the pre-defined trigger event is an inclement weather event at a target location.

13. A computer program comprising instructions that when executed on a processor:
answers a telephone call from a caller;
prompts the caller to request a service that requires integration with an electronic health record (“EHR”) system; and
communicates with the EHR system and provides the requested service to the caller during the telephone call.

14. The computer program of claim 13 further comprising instructions, that when executed by the processor, prompt the caller using lifelike speech.

15. The computer program of claim 14 further comprising instructions, that when executed by the processor:
generate the lifelike speech using an artificial intelligence (“AI”) model that converts text into the lifelike speech; and
prompt the caller using the lifelike speech.

16. The computer program of claim 13 further comprising instructions, that when executed by the processor:
prompt the caller in a first language; and
in response to detecting a voice input provided by the caller in a second language, interact with the caller in the second language.

17. The computer program of claim 13 further comprising instructions, that when executed by the processor, present appointment data to the caller using a first communication channel and a second communication channel.

18. An automated system for scheduling an appointment during a phone call, the automated system comprising:
an interactive voice response (“IVR”) system configured to receive a voice input from a caller; and
a middleware system configured to schedule an appointment for the caller based on the voice input.

19. The automated system of claim 18 wherein the middleware system is configured to present an available appointment slot to the caller based on a set of filtering rules.

20. The automated system of claim 19 wherein the filtering rules are dynamically set by an AI model based on a real-time availability of appointment slots in an electronic health record (“EHR”) system.
